(19) United States
(12) Patent Application Publication
(10) Pub. No.: US 2012/0190871 A1
(43) Pub. Date: Jul. 26, 2012

(54) TRITERPENOID COMPOSITION OF ANTRODIA CINNAMOMEA, PREPARATION AND ANALYSIS METHOD THEREOF

(75) Inventors: YANG-CHANG WU, KAOHSIUNG CITY (TW); FANG-RONG CHANG, KAOHSIUNG CITY (TW); MEI-CHIN LU, PINGTUNG COUNTY (TW); YING-CHI DU, CHIAYI CITY (TW); TUNG-YING WU, KAOHSIUNG CITY (TW); YU-MING HSU, KAOHSIUNG CITY (TW)

(73) Assignee: KAOHSIUNG MEDICAL UNIVERSITY, KAOHSIUNG CITY (TW)

(21) Appl. No.: 13/351,775

(22) Filed: Jan. 17, 2012

(30) Foreign Application Priority Data
Jan. 26, 2011 (TW) 100102927

(51) Int. Cl. C07J 9/00; G01R 33/44; G01N 30/00

(52) U.S. Cl. 552/541; 552/542; 73/61.52; 324/307

(57) ABSTRACT

Disclosed are the isolation, purification and analysis of the triterpenoid compositions (including ergostane and lanostane) in the fruiting body of Antrodia cinnamomea using HPLC and NMR, as well as the stereo structures and the amounts of the triterpenoid compositions. The cytotoxicity of triterpenoids is also revealed. Based on the aforementioned techniques, the presence and amounts of ergostane and lanostane in the drugs, healthcare food or other goods are able to be detected.

### CROSS-REFERENCE TO RELATED APPLICATION AND CLAIM OF PRIORITY

The application claims the benefit of Taiwan Patent Application No. 100102927, filed on Jan. 26, 2011, in the Taiwan Intellectual Property Office, the disclosures of which are incorporated herein in their entirety by reference.

### FIELD OF THE INVENTION

The present invention relates to a composition of the fruiting body of Antrodia cinnamomea (abbreviated as A. cinnamomea or AC). In particular, the present invention relates to a triterpenoid composition of the fruiting body of AC and the preparation method and the analytic method thereof.

### BACKGROUND OF THE INVENTION

Antrodia cinnamemea (AC), by name niu-chang-chih or jang-jy is an endemic fungus in Taiwan and grows in the internal heartwood (or the dark/humid wood surface) of the particular Cinnamomum kanehirai in 400 to 2000 meters altitude. Therefore, it is uneasily to find out the wide fruiting body of AC or identify the morphological appearance of this Aphyllophorales fungus. In addition, the price of AC is still high due to their biologically active components having potential pharmaceutical value.

Since the fruiting body of AC cannot be easily found and be artificially cultured, mycelia products of AC are popular in the market and announce to own anticancer activity, reduced treatment-related symptoms and other side effects. In addition, mycelia products of AC have recently been reported to have anti-oxidant, antihypersensitive and immunostimulatory effects (Liu et al., 2007). It has been claimed of these mycelia products that they contain active components similar to the wild fruiting bodies with cytotoxic triterpenes, steroids, as well as immunostimulatory polysaccharides reported previously (Chen et al., 1995; Yang et al., 1996).

Traditionally AC has been used as health food to prevent inflammation, hypertension, itchy skin and liver cancer. Therefore, extracts of mycelia and fruiting body of AC are deemed as a potential chemotherapeutic agent against hepatoma, as well as prostate, bladder, lung cancer cells and so on (Chen et al., 2007; Hsu et al., 2007; Peng et al., 2007; Song et al., 2005; Wu et al., 2006). However, the chemical distribution and pharmacological research of niu-chang-chih products are not clarified up to now.

In addition, Taiwan Patent No. 1299665 discloses the extract of AC and the preparation thereof, in which the mycelia of AC is extracted with ethanol to obtain polysaccharides for inhibiting matrix metalloproteinase activities. However, the extract is not extracted with the fruiting body of AC, and the mycelia product thereof cannot inhibit cancer cell growth. Taiwan Patent No. I279439 discloses that the mycelia of AC is cultured to obtain the cultured products by adjusting pH value of medium. However, there is no extraction method disclosed. Taiwan Patent No. 591110 discloses that γ-aminobutyric acid is extracted from the lyophilized mycelia of AC with water or organic solvents. However, the above-mentioned inventions did not disclose any product of the fruiting body of AC extracted with water or organic solvent, and there is no targeted second metabolites contained in the AC being identified.

It is therefore attempted by the applicant to deal with the above situation encountered in the prior art.

### SUMMARY OF THE INVENTION

In order to overcome the problems that compositions in the fruiting bodies (or mycelia) of A. cinnamomea or the extracts of AC cannot be efficiently isolated and the stereo structures of the compositions cannot be determined in the prior art, ergostane and lanostane triterpenoid compositions in the fruiting body of AC are isolated, purified and analyzed using techniques such as high performance liquid chromatography (HPLC), nuclear magnetic resonance (NMR) and so on in the present invention and their stereo structural formulas and amounts there in are determined. Based on the aforementioned techniques, it can be determined whether the ergostane and lanostane triterpenoid compositions are present in the medicines, healthcare food or other products and their amounts therein.

The present invention provides a pharmaceutical composition including at least one ergostane triterpenoid composition being represented by one selected from a group consisting of the formulas I˜VI, VIII˜IX and a combination thereof as described as following paragraphs.

The ergostane triterpenoid composition is extracted from the ethyl acetate (EA) extract of the fruiting body of A. cinnamomea (abbreviated as “EA extract”). In order to obtain the EA extract, the fruiting body of AC is sequentially extracted with the ethanol solution, the n-hexane solution and the EA solution. The ergostane triterpenoid composition is cytotoxic to leukemia cells.

The present invention further provides a method for preparing the ergostane triterpenoid composition, including a step of chromatographing the EA extract to obtain an ergostane triterpenoid composition including compositions (or named stearoisomeric pure compounds) having formulas I to X as described as follows.

Furthermore, the chromatographing step further includes a step of isolating the ergostane triterpenoid composition to obtain the stereoisomer by using HPLC column and under a condition of a solvent of acetonitrile and acid-containing water in a mobile phase.

Additionally, a lanostane triterpenoid composition is further isolated in the chromatographing step. The ergostane triperpenoid composition includes zhankuic acid A, B and C, antcin A, C and K, and the lanostane triterpenoid composition includes dehydrosulphurenic acid (formula XI), sulphurenic acid (formula XII), 15α-acetyl-dehydrosulphurenic acid (formula XIII), versisponic acid D (formula XIV), dehydroeburicoic acid (formula XV) and/or eburicoic acid (formula XVI) as described as follows.

The present invention further includes a method for detecting the amount of a stereoisomer of at least one ergostane triterpenoid composition in the fruiting body of AC, and the method includes steps of: extracting from the fruiting body the EA extract; detecting the EA extract by using ¹H NMR to identify whether the at least one ergostane triterpenoid composition is present in the EA extract; and detecting the amount of the stereoisomer of the at least one ergostane triterpenoid composition in the EA extract by using HPLC when the at least one ergostane triterpenoid composition is present in the EA extract.

Furthermore, the method further includes a step of detecting the methylene signal at C-28 position of the at least one ergostane triterpenoid composition by using ¹H NMR.

Additionally, the detection method is further used to detect the amount of the at least one lanostane triterpenoid composition, and the method includes steps of: detecting the EA extract by using ¹H NMR to identify whether the at least one lanostane triterpenoid composition is present in the EA extract; and detecting the amount by using HPLC when the at least one lanostane triterpenoid composition is present in the EA extract. The ¹H NMR detection is to detect the methylene signal at C-28 position of the at least one lanostane triterpenoid composition, and HPLC uses the detector including the full wavelength detector, the single wavelength detector and/or the tandem mass spectrometer.

The present invention further provides a method for isolating a stereoisomer of a compound having a pKa value and an asymmetrical center at an α-position of a carboxylic group. The method includes steps of: calculating the pKa value being represented by a symbol A; adjusting a pH value of a separating solvent to have a value B ranged at A−1.5≦B≦A+1.5 and 1.0≦B≦7; and chromatographing the compound by using the separating solvent to isolate the stereoisomer.

The present invention further provides a method for detecting the amount of the ergostane triterpenoid composition having a methylene signal at a C-28 position in an extract. The method includes steps of: preparing a NMR spectrum and a calibration curve based on zhankuic acid A samples with a various of concentrations; detecting the methylene signal at the C-28 position by using ¹H NMR; and comparing the calibration curve with the methylene signal at the C-28 position to calculate the amount by an integral area ratio of the methylene signal at the C-28 position.

Based on the aforementioned detection method, the present invention further provides a method for detecting the amount of the lanostane triterpenoid composition having a methylene signal at a C-28 position in an extract. The method includes steps of: preparing a NMR spectrum and a calibration curve based on dehydroeburicoic acid samples with a various of concentrations; detecting the methylene signal at the C-28 position by using ¹H NMR; and comparing the calibration curve with the methylene signal at the C-28 position to calculate the amount by an integral area ratio of the methylene signal at the C-28 position.

The present invention further provides a method for detecting a stereoisomer of an ergostane triterpenoid composition in the EA extract. The method includes steps of: chromatographing the EA extract by using HPLC column to isolate the stereoisomer; and determining R-form or S-form at a C-25 position of the stereoisomer according to ¹H NMR spectrum of the stereoisomer, retention time of the HPLC column and an optical rotation.

The above objectives and advantages of the present invention will become more readily apparent to those ordinarily skilled in the art after reviewing the following detailed descriptions and accompanying drawings.

### BRIEF DESCRIPTION OF THE DRAWINGS

FIG. 1 illustrates a flowchart showing a preparation method of the EA extract of the fruiting body of AC in the present invention.

FIG. 2 illustrates a recycle chromatography spectrum of HPLC showing the stereoisomeric mixture of zhankuic acid A.

FIG. 3 illustrates a chromatographic spectrum of pure compounds E9 and E10 isolated from the stereoisomeric mixture of zhankuic acid A.

FIG. 4 illustrates a chromatographic spectrum of pure compounds E3 and E4 isolated from the stereoisomeric mixture of antcin C.

FIG. 5 illustrates a chromatographic spectrum of pure compounds E5 and E6 isolated from the stereoisomeric mixture of zhankuic acid C.

FIGS. 6(a), 6(b) and 6(c) respectively illustrate the ¹H NMR spectra of (a) zhankuic acid A, (b) compound E9 and (c) compound E10 dissolved in C₅D₅N at 600 MHz.

FIGS. 7(a), 7(b) and (7c) respectively illustrate the ¹³C NMR spectra of (a) zhankuic acid A, (b) compound E9 and (c) compound E10 dissolved in C₅D₅N at 150 MHz.

FIGS. 8(a) and 8(b) respectively illustrate the diagrams showing the chemical structures of (a) a synthetic ester compound E9-1RAT and (b) a synthetic ester compound E9-1SAT.

FIG. 9 illustrates a diagram showing the absolute stereoisomer at C-25 position in accordance with the difference value of ¹H NMR chemical shifts between the synthetic ester compounds, (1R)- and (1S)-1-(9-anthryl)-2,2,2-trifluoroethanol, of ergostane triterpenoid composition.

FIG. 10 illustrates a HPLC spectrum of the EA extract at a wavelength of 254 nm using different organic acids (0.1% trifluoacetic acid, 0.1% formic acid and 0.1% acetic acid) as the mobile phase.

FIGS. 11(a) and 11(b) respectively illustrate the HPLC spectra of EA extract (a) in HPLC method 1 and (b) in HPLC method 2.

FIGS. 12(a) and 12(b) respectively illustrate the comparisons of HPLC spectra of the EA extract that (a) pH value is adjusted to 3.75 and 4.0 using ammonium acetate and (b) pH value is adjusted to 4.25, 4.5 and 5.0 using ammonium acetate at the mobile phase of 0.1% acetic acid (pH 3.3) and the detection wavelength of 254 nm.

FIG. 13 illustrates a HPLC spectrum showing the compounds represented by the peaks at the optimal analytic conditions.

FIGS. 14(a), 14(b), 14(c), 14(d), 14(e) and 14(f) respectively illustrate the HPLC spectra showing (a) compounds E1, E2 and antcin K, (b) compounds E3, E4 and antcin C, (c) compounds E5, E6 and zhankuic acid C, (d) compounds E7, E8 and zhankuic acid B, (e) compounds E9, E10 and zhankuic acid A, and (f) compounds E11, E12 and antcin A at a wavelength of 254 nm.

FIGS. 15(a) and 15(b) respectively illustrate (a) a ¹H NMR spectrum of the EA extract and the internal standard (pyrazine) dissolved in DMSO-d6 at 400 MHz, and (b) a magnification spectrum showing the C-28 methylene characteristic signals of zhankuic acid A and dehydroeburicoic acid.

### DETAILED DESCRIPTION OF THE PREFERRED EMBODIMENT

The present invention will now be described more specifically with reference to the following Embodiments. It is to be noted that the following descriptions of preferred Embodiments of this invention are presented herein for purpose of illustration and description only; it is not intended to be exhaustive or to be limited to the precise form disclosed.

### Embodiments

For conveniently describing the ergostane triterpenoid compositions E1 to E 12 extracted in the present invention, compositions E1 to E12, the corresponding structural formulas (Formulas I to X) and the corresponding peaks in the HPLC spectra were detailedly illustrated as follows.

| Ergostane | | Structural | | |
|---|---|---|---|---|
| triterpenoid | Source | formula | Peak | IUPAC nomination |
| E1 | antcin K | I | 1 | 3α,4β,7β-trihydroxy-4α-methylergosta- 8,24(28)-dien-11-on-25S-26-oic acid |
| E2 | | II | 2 | 3α,4β,7β-trihydroxy-4α-methylergosta- 8,24(28)-dien-11-on-25R-26-oic acid |
| E3 | antcin C | III | 3 | 7β-hydroxy-4α-methylergosta-8,24(28)- dien-3,11-dion-25S-26-oic acid |
| E4 | | IV | 4 | 7β-hydroxy-4α-methylergosta-8,24(28)- dien-3,11-dion-25R-26-oic acid |
| E5 | zhankuic acid C | V | 5 | 3α, 12α-dihydroxy-4α-methylergosta- 8,24(28)-dien-7,11-dion-25S-26-oic acid |
| E6 | | VI | 6 | 3α, 12α-dihydroxy-4α-methylergosta- 8,24(28)-dien-7,11-dion-25R-26-oic acid |
| E7 | zhankuic acid B | VII | 8 | 3α-hydroxy-4α-methylergosta-8,24(28)- |
| E8 | | | 9 | dien-7,11-dion-26-oic acid |
| E9 | zhankuic acid A | VIII | 10 | 4α-methylergosta-8,24(28)-dien-3,7,11- trion-25S-26-oic acid |
| E10 | | IX | 11 | 4α-methylergosta-8,24(28)-dien-3,7,11- trion-25R-26-oic acid |
| E11 | antcin A | X | 12 | 4α-methylergosta-8,24(28)-dien-3,11- |
| E12 | | | 13 | dion-26-oic acid |

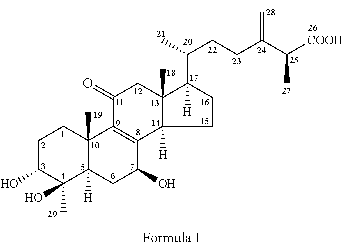

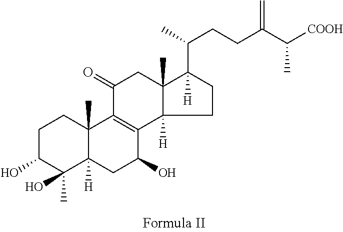

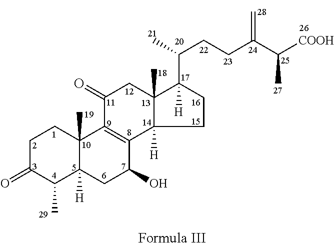

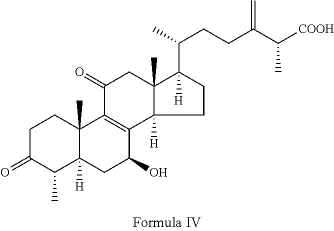

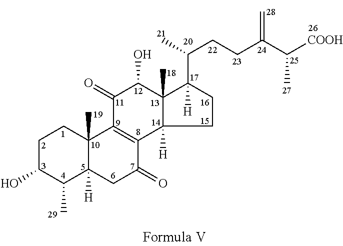

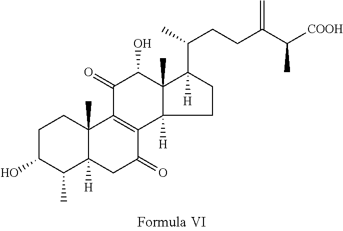

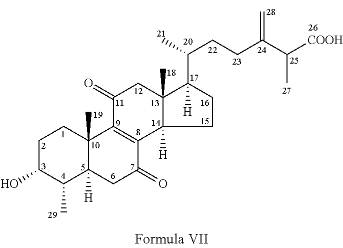

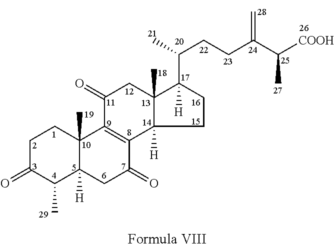

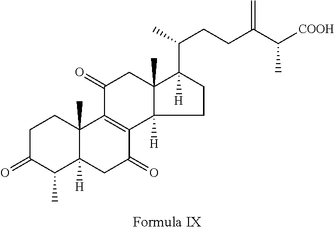

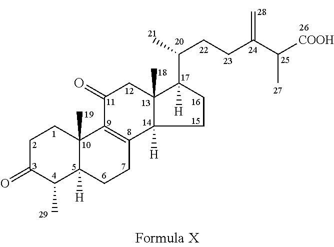

For conveniently describing the lanostane triterpenoid compositions L1 to L6 extracted in the present invention, compounds L1 to L6, the corresponding structural formulas (Formulas XI to XVI) and the corresponding peaks in the HPLC spectra were detailedly illustrated as follows.

| Lanostane | Structural | | |
|---|---|---|---|
| triterpenoid | formula | Peak | Nomination |
| L1 | XI | 7 | dehydrosulphurenic acid |
| L2 | XII | | sulphurenic acid |
| L3 | XIII | 14 | 15α-acetyl- dehydrosulphurenic acid |
| L4 | XIV | | versisponic acid D |
| L5 | XV | 15 | dehydroeburicoic acid |
| L6 | XVI | 16 | eburicoic acid |

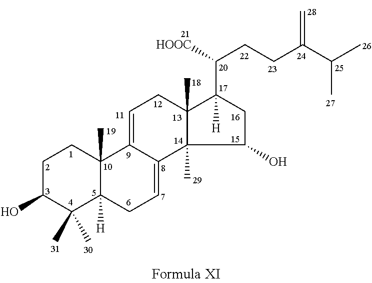

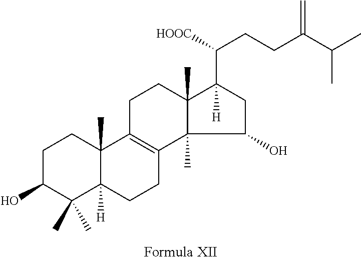

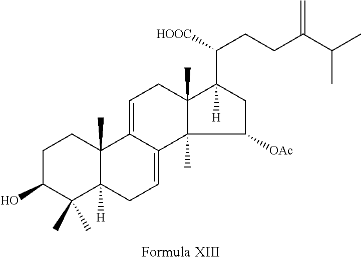

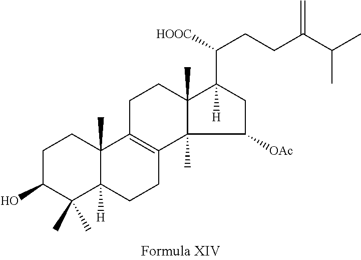

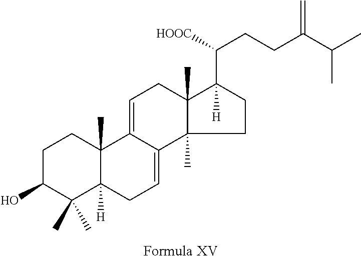

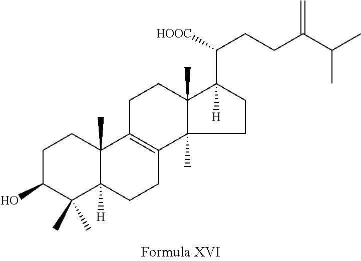

### Experiment 1

### Preparation of the EA Extract of the Fruiting Body of AC

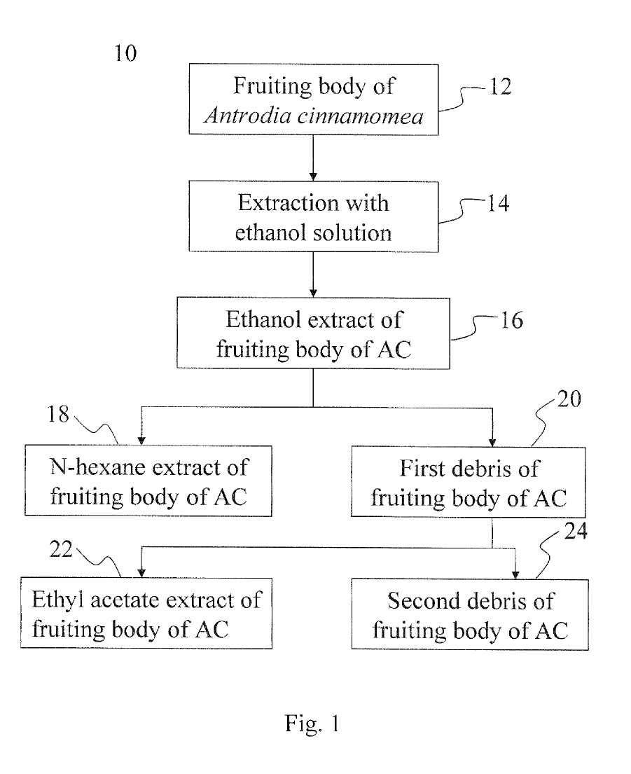

Please refer to preparation method 10 in FIG. 1, the dried fruiting body of AC was ground as fine powder (step 12), which was heated at reflux in ethanol (EtOH) solution at 75° C. at a ratio of 1/10 (weight/volume) for 2 hours (step 14). The extract was cooled, and then was precipitated at 4° C. overnight. Furthermore, the supernatant of the extract was filtered with filter paper, and the precipitate was removed by centrifuging at 3,000 rpm for 30 min. The extract, which was the EtOH extract of the fruiting body of AC, was lyophilized and stored at −70° C. (step 16). The EtOH extract was extracted with n-hexane to obtain the n-hexane extract of the fruiting body of AC (step 18) and the first debris of the fruiting body of AC (step 20).

Next, the first debris (step 20) was extracted with ethyl acetate (EA) to obtain the EA extract of the fruiting body of AC (hereinafter “the EA extract”, step 22) and the second debris of the fruiting body of AC (step 24).

### Experiment 2

### Isolation of Ingredients of Ergostane Triterpenoids

The EA extract (6.8 g) was chromatographed in gradient with n-hexane-EtOAc-methanol (MeOH) (sequentially 10:1:0, 5:1:0, 1:1:0, 0:1:0, 0:40:1, 0:30:1, 0:20:1, 0:10:1) with Silica gel 60 (Merck, 230-400 mesh) to obtain 17 fractions.

(1) Isolation of antcin K: Fraction 15 (245.7 mg) was purified with ODS HPLC column (250×10 mm, Hypersil ODS, acetonitrile (CH₃CN)—H₂O (0˜2 min (35% CH₃CN˜45% CH₃CN); 20˜25 min (45% CH₃CN˜100% CH₃CN)) to afford antcin K (retention time of 14.7 min, flow rate of 3 ml/min).

(2) Isolation of antcin C: Fraction 10 (132.6 mg) was isolated using thin layer chromatography (TLC) with dichloromethane (CH₂Cl₂)-MeOH (15:1), and the chromatographic band with Rf value of 0.31 was harvested and then purified with ODS HPLC column (250×10 mm, Hypersil®, CH₃CN—H₂O (70:30)) to afford antcin C (retention time of 10 min, flow rate of 2 ml/min).

(3) Isolation of zhankuic acid C: Fraction 13 (100.0 mg) was isolated using TLC with CH₂Cl₂-MeOH (15:1), and the chromatographic band with Rf value of 0.18 was harvested and then purified with ODS HPLC column (250×10 mm, Hypersil®, CH₃CN—H₂O (70:30)) to afford zhankuic acid C (retention time of 10 min, flow rate of 2 ml/min).

(4) Isolation of zhankuic acid B: Fraction 10 (132.6 mg) was isolated using TLC with CH₂Cl₂-MeOH (15:1), and the chromatographic band with Rf value of 0.31 was harvested and then purified with ODS HPLC column (250×10 mm, Hypersil®, CH₃CN—H₂O (50:50)) to afford zhankuic acid B (retention time of 50 min, flow rate of 2 ml/min).

(5) Isolation of zhankuic acid A: Fraction 6 (100.0 mg) was isolated using TLC with CH₂Cl₂-MeOH (15:1), and the chromatographic band with Rf value of 0.42 was harvested and then purified with ODS HPLC column (250×10 mm, Hypersil®, CH₃CN—H₂O (75:25)) to afford zhankuic acid A (retention time of 12 min, flow rate of 2 ml/min).

(6) Isolation of antcin A: Fraction 6 (100.0 mg) was isolated using TLC with CH₂Cl₂-MeOH (15:1), and the chromatographic band with Rf value of 0.42 was harvested and then purified with ODS HPLC column (250×10 mm, Hypersil®, CH₃CN—H₂O (75:25)) to afford antcin A (retention time of 19 min, flow rate of 2 ml/min).

### Experiment 3

### Isolation of Ingredients of Lanostane Triterpenoids

(1) Isolation of dehydrosulphurenic acid: Fraction 13 (200.0 mg) was isolated twice using TLC with CH₂Cl₂-MeOH (15:1), and the chromatographic band with Rf value of 0.36 was harvested and then purified with ODS HPLC column (250×10 mm, Hypersil®, CH₃CN—H₂O (60:40)) to afford dehydrosulphurenic acid (retention time of 22 min, flow rate of 2 ml/min).

(2) Isolation of sulphurenic acid: Fraction 10 (132.6 mg) was isolated using TLC with CH₂Cl₂-MeOH (15:1), and the chromatographic band with Rf value of 0.31 was harvested and then purified with ODS HPLC column (250×10 mm, Hypersil®, CH₃CN—H₂O (50:50)) to afford sulphurenic acid (retention time of 53 min, flow rate of 2 ml/min).

(3) Isolation of 15α-acetyl-dehydrosulphurenic acid: Fraction 6 (100.0 mg) was isolated using TLC with CH₂Cl₂-MeOH (15:1), and the chromatographic band with Rf value of 0.42 was harvested and then purified with ODS HPLC column (250×10 mm, Hypersil®, CH₃CN—H₂O (75:25)) to afford 15α-acetyl-dehydrosulphurenic acid (retention time of 20 min, flow rate of 2 ml/min).

(4) Isolation of versisponic acid D: Fraction 6 (100.0 mg) was isolated using TLC with CH₂Cl₂-MeOH (15:1), and the chromatographic band with Rf value of 0.42 was harvested and then purified with ODS HPLC column (250×10 mm, Hypersil®, CH₃CN—H₂O (75:25)) to afford versisponic acid D (retention time of 22 min, flow rate of 2 ml/min).

(5) Isolation of dehydroeburicoic acid: Fraction 5 (100.0 mg) was purified with ODS HPLC column (250×10 mm, Hypersil®, CH₃CN—H₂O (90:10)) to afford dehydroeburicoic acid (retention time of 27 min, flow rate of 2 ml/min).

(6) Isolation of eburicoic acid: Fraction 5 (100.0 mg) was purified with ODS HPLC column (250×10 mm, Hypersil®, CH₃CN—H₂O (90:10)) to afford eburicoic acid (retention time of 31 min, flow rate of 2 ml/min).

### Experiment 4

### Isolation of Ergostane Triterpenoid Stereoisomeric Mixtures Having Asymmetrical Centers

At present, there is no prior art or literature to disclose the absolute stereo structure of ergostane triterpenoid compositions, and no pure compound is obtained. Based on the following descriptions, the present invention is the first technical literature in the world to disclose the asymmetrical center at C-25 position of ergostane triterpenoid composition, and pure compounds were obtained.

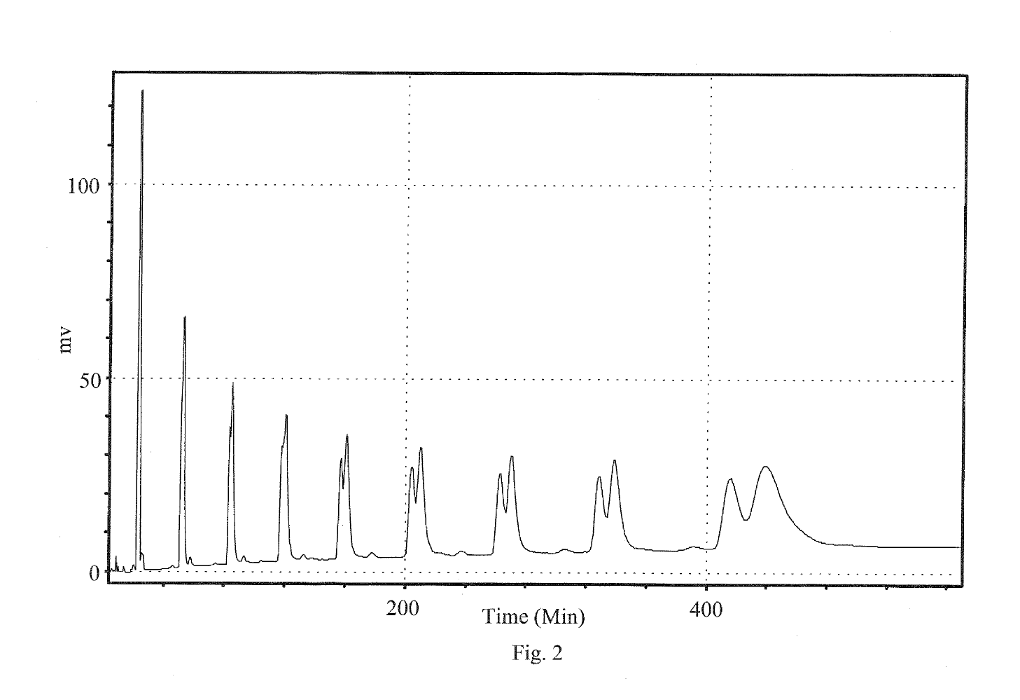

Taking the isolation of stereoisomeric mixture of zhankuic acid A as the example, the zhankuic acid A standard obtained in Experiment 2 showed a spot in the positive phase TLC (solvent system is CH₂Cl₂-MeOH (20:1)) for one separation. However, other closer spots were found after a various of separations, and the phenomenom that stereoisomeric mixtures were separated was observed. Please refer to FIG. 2, which illustrates the recycle chromatography spectrum using reverse phase HPLC. Purification was performed using ODS HPLC column (250×10 mm, Hypersil®, CH₃CN—H₂O (55:45), flow rate of 4.3 ml/min). After the eighth recycle separation, the stereoisomeric mixtures of zhankuic acid A were separated at retention time of 416 and 447 min, and pure compounds E9 and E10 were afforded respectively. The stereoisomeric mixtures of other ergostane triterpenoids in Experiment 2 could be separated using the same method.

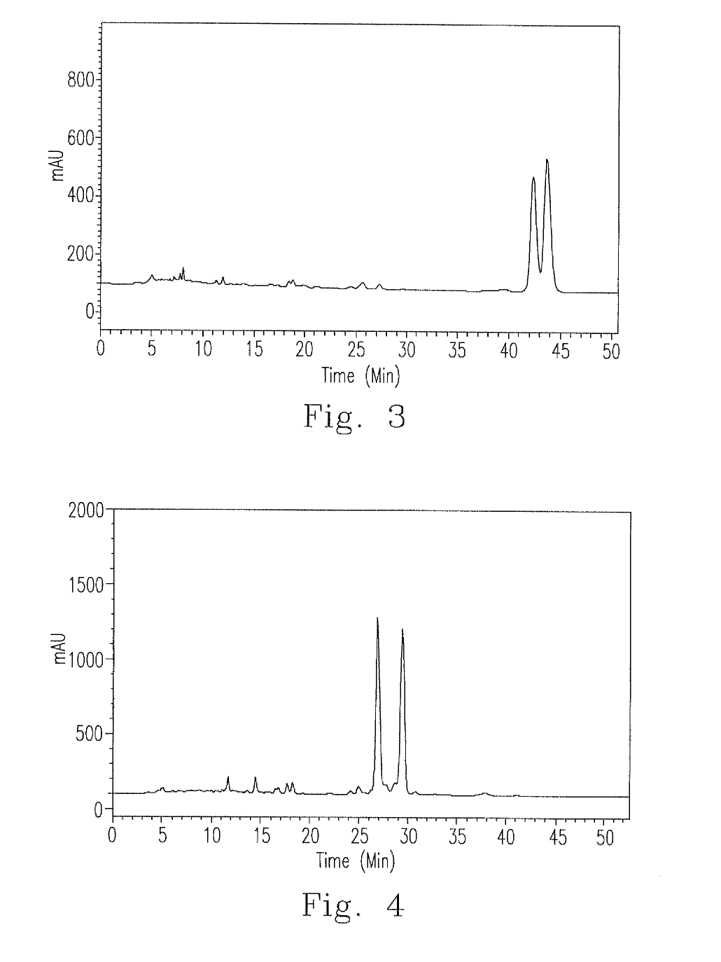

Please refer to FIG. 3, in addition to the above method, the stereoisomeric mixtures of zhankuic acid A were separated using HPLC Cosmosil 5C-18-MS column (250×10.0 mm) at retention time of 42 and 43 min at the conditions that solvents A and B respectively were CH₃CN and H₂O (containing 0.05% acetic acid) in the mobile phase and solvent system was CH₃CN—H₂O (50:50) at the flow rate of 3.0 ml/min, and pure compounds E9 and E10 were afforded respectively.

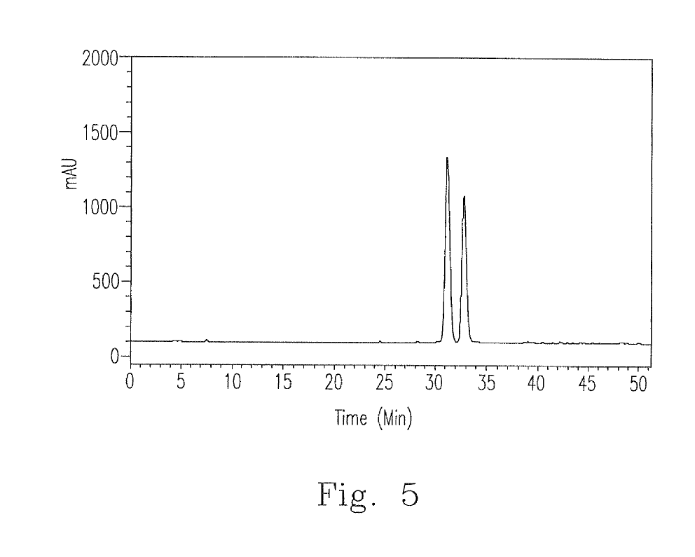

The stereoisomeric mixtures of other ergostane triterpenoids also were separated at the condition of mobile phase containing acid. The stereoisomeric mixtures of antcin K were purified using ODS HPLC column (250×10 mm, Hypersil®, CH₃CN—H₂O (0˜2 min (35% CH₃CN˜45% CH₃CN); 20˜25 min (45% CH₃CN˜100% CH₃CN) at retention time of 14.5 and 15.3 min, and pure compounds E1 and E2 were afforded respectively. Please refer to FIG. 4, antcin C was purified using Cosmosil HPLC column (250×10 mm, CH₃CN—H₂O (50:50), flow rate of 3.0 ml/min), the stereoisomeric mixtures of antcin C were separated at retention time of 27 and 29 min, and pure compounds E3 and E4 were afforded respectively. Please refer to FIG. 5, zhankuic acid C was purified using Cosmosil HPLC column (250×10 mm, CH₃CN—H₂O (50:50), flow rate of 3.0 ml/min), the stereoisomeric mixtures of zhnakuic acid C were separated at retention time of 31 and 33 min, and pure compounds of E5 and E6 were afforded respectively. Zhnakuic acid B was purified using Cosmosil HPLC column (250×10 mm, CH₃CN—H₂O (0˜20 min (55% CH₃CN˜60% CH₃CN); 20˜25 min (60% CH₃CN˜100% CH₃CN), flow rate of 3.0 ml/min), the stereoisomeric mixtures of zhnakuic acid B were separated at retention time of 19.84 and 20.29 min, and pure compounds E7 and E8 were afforded respectively. Antcin A was purified using Cosmosil HPLC column (250×10 mm, CH₃CN—H₂O (60:40), flow rate of 3.0 ml/min), the stereoisomeric mixtures of antcin A were separated at retention time of 32.73 and 33.83 min, and pure compounds E11 and E12 were afforded respectively.

### Experiment 5

### Structural Identification of Ergostane Triterpenoid Stereoisomers Having Asymmetrical Centers

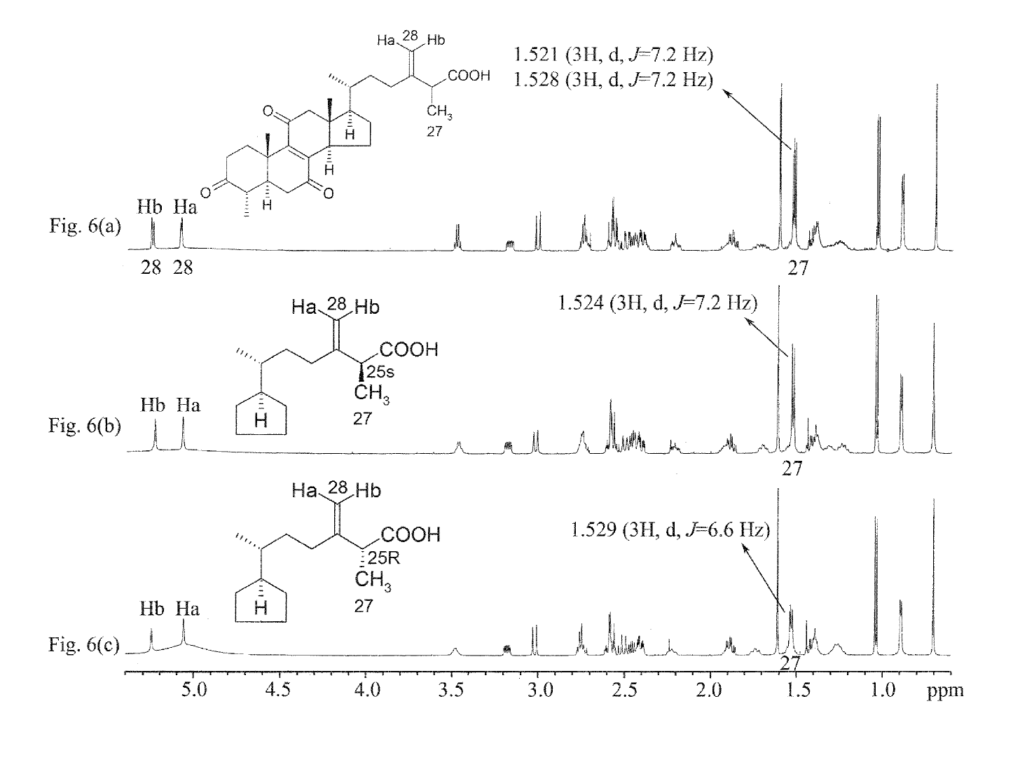

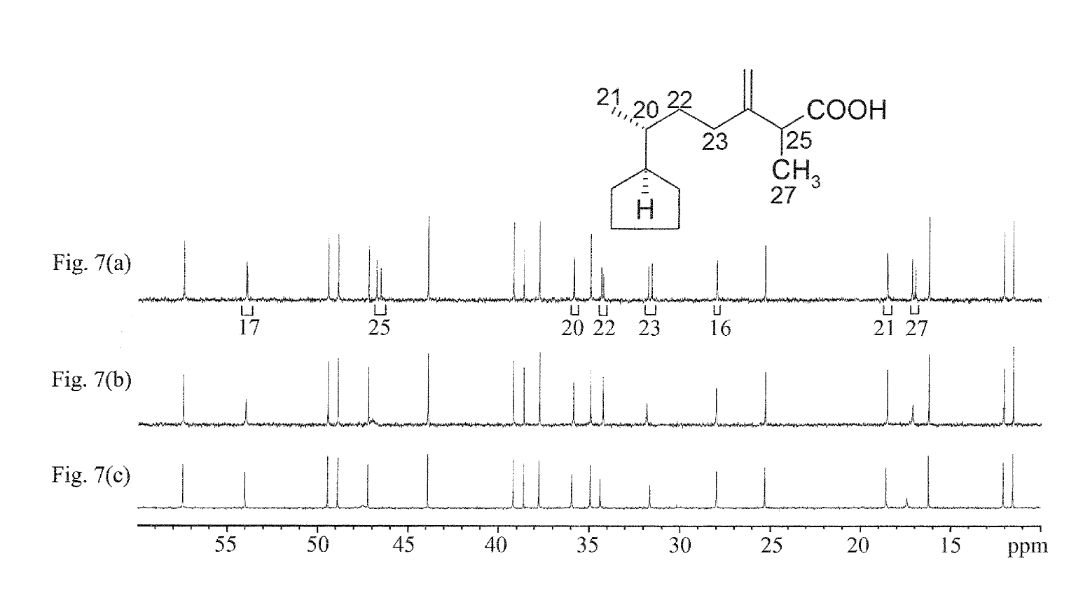

Based the separation method in Experiment 4, six ergostane triterpenoid stereoisomeric mixtures were separated and purified and 12 pure compounds E1 to E12 were afforded. Taking structural identification of the afforded compounds E9 and E10 separated from zhankuic acid A as the example, zhankuic acid A is a stereoisomeric mixture having an asymmetrical center at C-25 position at its structure. Please refer to FIG. 6(a), zhankuic acid A showed two sets of CH₃-27 signal at δ_{H}1.521 (3H, d, J=7.2 Hz) and 1.528 (3H, d, J=7.2 Hz) in ¹H NMR spectrum. Please refer to FIG. 7(a), zhankuic acid A significantly showed two sets of signals at δ_{C} 34.242 and 34.342 (CH₂-22), 31.575 and 31.766 (CH₂-23), 46.558 and 46.793 (CH-25), and 17.003 and 17.179 (CH₃-27) at side chains in ¹³C NMR spectrum (150 MHz in C₅D₅N) due to the C-25 asymmetrical center. Two sets of signals at δ_{C} 27.960 and 27.997 (CH₂-16), 53.937 and 53.986 (CH-17), 35.847 and 35.885 (CH-20), and 18.519 and 18.564 (CH₃-21) also could be observed.

Please refer to FIGS. 6(b), 6(c), 7(b) and 7(c), the afforded compounds E9 and E10 separated from zhankuic acid A only showed one set of signal in NMR spectrum, but there was no two-set characteristic signal of stereoisomer mixture presented. With the comparisons of the above NMR spectra, it was proved that the stereoisomeric mixtures of zhankuic acid A were respectively separated and purified, and pure compounds were afforded.

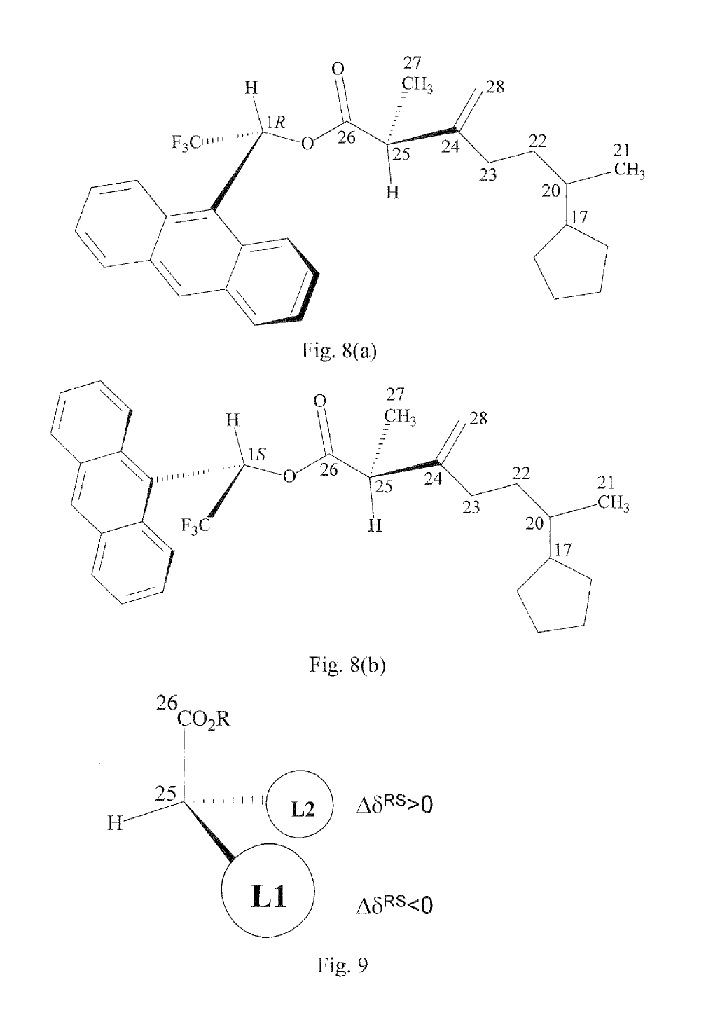

Please refer to FIG. 8, C-26 carboxylic group of compound E9 formed an ester with C-1 position (R-form) of (1R)-1-(9-anthryl)-2,2,2-trifluoroethanol (1RAT). Please refer to FIG. 8(b), compound E9 formed an ester with C-1 position (S-form) of (1S)-1-(9-anthryl)-2,2,2-trifluoroethanol (1SAT). Then, the absolute stereo structure of compound E9 at C-25 position was determined by the difference of ¹H NMR chemical shifts (Δδ^{RS} values in FIG. 9) between the synthetic compounds E9-1RAT and E9-1SAT. Please refer to FIG. 9, it was supposed that group L1 which had a negative signal difference value between the 1RAT and 1SAT synthetic esters was disposed over a horizontal plane and group L2 which a positive signal difference value therebetween was disposed below the horizontal plane. The absolute stereo structure of compound E9 at C-25 position then was determined by Cahn-Ingold-Prelog priority rules.

The experimental method was described as follows. Compound E9 (6.42 mg) was mixed with 1RAT (1 equivalent) and dissolved in tetrahydrofuran (THF) to obtain solution A. 1-Ethyl-3-(3-dimethylaminopropyl)carbodiimide (EDC-HCl, 3 equivalents) was mixed with 4-dimethylaminopyridine (DMAP, 1.5 equivalents) and dissolved in CH₂Cl₂ to obtain solution B. Solutions A and B were mixed, and then triethylamine (Et₃N, 2 equivalents) was added to react for 12 hours. Partition extraction was performed with H₂O and CH₂Cl₂, and the obtained organic layer was analyzed with pre-TLC and separated with CH₂Cl₂, to obtain compound E9-1RAT (3.43 mg). The ¹³C NMR signals of compounds E9 and E9-1RAT at C-26 position respectively were δ_{C} 176.900 and 172.774, indicating that compound E9 successfully formed an ester bond with 1RAT at C-26 position.

Compound E9-1SAT was also obtained with the similar reaction steps. Compound E9 (11.15 mg) was mixed with 1SAT (1 equivalent) to cooperatively dissolved in THF to obtain solution A. EDC-HCl (3 equivalents) was mixed with DMAP (1.5 equivalents) to cooperatively dissolved in CH₂Cl₂ to obtain solution B. Solutions A and B were mixed, and then Et₃N (2 equivalents) was added to react for 12 hours. Partition extraction was performed with H₂O and CH₂Cl₂, and the obtained organic layer was analyzed with pre-TLC and separated with CH₂Cl₂, to obtain an ester compound E9-1SAT (9.01 mg), which showed a C-26 ester signal of δ_{C} 172.681.

Please refer to Table 7, the difference value of ¹H NMR chemical shifts between compounds E9-1RAT and E9-1SAT was positive (Δδ^{RS}>0) at C-27 position and was negative (Δδ^{RS}<0) at C-28 position. It was determined that C-25 position of compound E9 was S form. Compound E9 was nominated as 4α-methylergosta-8,24(28)-dien-3,7,11-trion-25S-26-oic acid, and its NMR data was referred to Table 4.

Compound E10 of 7.73 mg and 9.17 mg respectively were esterified with 1RAT and 1SAT, and partition extraction was performed with H₂O and CH₂Cl₂ beyond the reaction. The obtained organic layer was analyzed with pre-TLC and separated with CH₂Cl₂, to obtain compounds E10-1RAT (5.44 mg) and E10-1SAT (9.86 mg). Please refer to Table 7, the difference value of ¹H NMR chemical shifts between compounds E10-1RAT and E10-1SAT was negative (Δδ^{RS}<0) at C-27 position and was positive (Δδ^{RS}>0) at C-28 position. It was determined that C-25 position of compound E10 was R form. Compound E10 was nominated as 4α-methylergosta-8,24(28)-dien-3,7,11-trion-25R-26-oic acid, and its NMR data was referred to Table 4.

After determining the absolute configuration, we tried to measure the optical rotation aiming to complete the spectral profile for the isolated compounds. In a previous report, the mixture form of zhankuic acid A was dissolved in methanol to measure its optical rotation. However, the solubility of E9 (25S) and E10 (25R) was not sufficient in single solvent (methanol or ethanol). The solubility of compounds E9 and E10 improved through using acetone-methanol mixture. Considering compounds solubility and the convenience of collecting physical and spectral data using single solvent, pyridine was used in optical rotation experiment. The optical rotation of compound E9 was [α]_{D}²⁵+32.1 (c 0.70, pyridine), and that of compound E10 was [α]_{D}²⁵+9.0 (c 0.84, pyridine).

The optical rotations of compounds E3 and E4, which were obtained by isolating from stereoisomeric mixture of antcin C, respectively were [α]_{D}²⁵+124.8 (c 0.81, pyridine) and [α]_{D}²⁵+79.9 (c 0.47, pyridine). ¹H NMR spectrum characteristic signals of 1RAT and 1SAT eater compounds of compounds E3 and E4 were described in Table 5. Based on the difference value of ¹H NMR chemical shifts at C-27 and C-28 positions beyond reaction, it was determined that C-25 position of compound E3 was S form. Compound E3 was nominated as 7β-hydroxy-4α-methylergosta-8,24(28)-dien-3,11-dion-25S-26-oic acid, and its NMR data was referred to Table 2. C-25 position of compound E4 was R form. Compound E4 was nominated as (7β-hydroxy-4α-methylergosta-8,24(28)-dien-3,11-dion-25R-26-oic acid, and its NMR data was referred to Table 2.

Compounds E5 and E6 were obtained by isolating from stereoisomeric mixture of zhankuic C, and their optical rotations were [α]_{D}²⁵+82.0 (c 0.64, pyridine) and [α]_{D}²⁵+110.6 (c 0.70, pyridine). ¹H NMR spectrum characteristic signals of 1RAT and 1SAT ester compounds of compounds E5 and E6 were described in Table 6. Based on the difference value of ¹H NMR chemical shifts at C-27 and C-28 positions beyond reaction, it was determined that C-25 position of compound E5 was R form. Compound E5 was nominated as 3α,12α-dihydroxy-4α-methylergosta-8,24(28)-dien-7,11-dion-25R-26-oic acid, and its NMR data was referred to Table 3. C-25 position of compound E6 was S form. Compound E6 was nominated as 3α,12α-dihydroxy-4α-methylergosta-8,24(28)-dien-7,11-dion-25S-26-oic acid, and its NMR data was referred to Table 3.

Compounds E1 and E2 were obtained by isolating from stereoisomeric mixture of antcin K, and their optical rotations respectively were [α]_{D}²⁵+61.0 (c 0.42, pyridine) and [α]_{D}²⁵+71.8 (c 0.27, pyridine). Since the amount of the obtained compound E1 was insufficient, only compound E2 was used to proceed esterification of 1RAT and 1SAT. The characteristic signals of ¹H NMR for compound E2-1RAT was δ_{H} 1.342 (CH₃-27, d, J=7.2 Hz) and 5.170, 5.118 (CH₂-28), and those for compound E2-1SAT was δ_{H} 1.387 (CH₃-27, d, J=7.2 Hz) and 4.903, 5.005 (CH₂-28). The difference value of ¹H NMR chemical shifts at C-27 position was negative (Δδ^{RS}<0) and that at C-28 position was positive (Δδ^{RS}>0), and it was determined that C-25 position of compound E2 was R form. Compound E2 was nominated as 3α,4α,7α-trihydroxy-4α-methylergosta-8,24(28)-dien-11-on-25R-26-oic acid, and its NMR data was referred to Table 1. C-25 position of compound E1 was S form. Compound 1 was nominated as 3α,4α,7α-trihydroxy-4α-methylergosta-8,24(28)-dien-11-on-25S-26-oic acid, and its NMR data was referred to Table 1.

In addition to the above major ergostane triterpenoid components, the minor ergostane triterpenoid stereoisomeric mixtures also were isolated and purified. The optical rotations of compounds E7 and E8, which were isolated from stereoisomeric mixture of zhankuic acid B, respectively were [α]_{D}²⁵+11.9 (c 0.57, pyridine) and [α]_{D}²⁵+36.4 (c 0.49, pyridine). The optical rotations of compounds E11 and E12, which were isolated from stereoisomeric mixture of antcin A, respectively were [α]_{D}²⁵+146.9 (c 0.69, pyridine) and [α]_{D}²⁵+117.2 (c 0.34, pyridine). Since the amounts of the obtained compounds E7, E8, E11 and E12 were insufficient, their 1RAT and 1SAT esterifications were not proceeded. It could be known from the HPLC spectrum and optical rotation data that the stereoisomeric mixtures with asymmetrical centers at C-25 position have been isolated, and was obtained in form of pure compounds.

### Experiment 6

### Cytotoxicity Test of Ergostane Triterpenoid Stereoisomeric Pure Compounds on Cancer Cells

The cytotoxicity test of the obtained major ergostane triterpenoid compounds (zhankuic acid A, zhankuic acid C, antcin C and antcin K) and their stereoisomeric pure compounds (compounds E1 to E6 and E9 to E10) were performed on three leukemia cell lines using MTT (3-(4,5-dimethylthiazol-2-yl)-2,5-diphenyltetrazolium bromide) colorimetric method, and the results were referred to Table 8.

### Experiment 7

### HPLC Analysis

The component analysis of the EA extract was further performed using HPLC method to set up the optimal analytic conditions, and ergostane triterpenoid stereoisomeric mixtures could be completely isolated and lanostane triterpenoid compounds could be detected simultaneously. What is mainly discussed in this experiment is described as follows. (1) The baseline stability and resolution relationship between the ergostane and lanostane acidic compounds in the chromatographic spectra are compared by adding different types of organic acids in the water mobile phase, and the most appropriate organic acid for adding in the water mobile phase is selected. (2) The acidity coefficient (pKa) of the test compound structure was calculated using analytic software, and average acidity coefficient of ergostane and lanostane acidic compounds are determined. Furthermore, the pH values of the mobile phase are monitored using pH meter to make the pH values of the mobile phase approach the average acidity coefficient of the compound, and thus the optimal separation effect can be achieved.

HPLC method 1. The detection method was described as follows. The EA extract (1.0 mg) was dissolved in 1 ml MeOH to be the sample for HPLC analysis. The conditions of HPLC were described as follows. HPLC apparatus was Shimadzu LC-10AT, detector was Shimadzu SPD-M10A photodiode array detector, auto sampler was Shimadzu SIL-20A prominence auto sampler, and HPLC column was Cosmosil 5C-18-MS (250×4.6 mm, 5 μm). Solvents A and B in the mobile phase respectively were CH₃CN and pure water (HPLC grade H₂O), and the various organic acids, 0.1% trifluoacetic acid (TFA, pH 2.20), 0.1% formic acid (pH 2.80) and 0.1% acetic acid (pH 3.30), were added respectively. Flow rate was 1 ml/min. The temperature of column was room temperature, and detection wavelength was UV 254 nm. The conditions of solvent system were described as follows. Mobile phase included solvents A and B, linear gradients were 0˜30 min (45% A˜50% A), 30˜35 min (50% A˜55% A), 35˜45 min (55% A˜60% A), 45˜55 min (60% A˜70% A), 55˜60 min (70% A˜85% A) and 60˜100 min (85% A˜100% A). Flow rate and column temperature were the same as above.

HPLC method 2. The detection method was described as follows. The EA extract (1.0 mg) was dissolved in 1 ml MeOH to be the sample for HPLC analysis. The conditions of HPLC were described as follows. HPLC apparatus was Shimadzu LC-10AT, detector was Shimadzu SPD-M10A photodiode array detector, auto sampler was Shimadzu SIL-20A prominence auto sampler, and HPLC column was Agilent Poroshell 120 SB-C18 (150×4.6 mm, 2.7 μm). Solvents A and B in the mobile phase were CH₃CN and pure water (HPLC grade H₂O, contain 0.1% acetic acid (pH 3.30)). Flow rate was 1.3 ml/min. The temperature of column was room temperature, and detection wavelength was UV 254 nm. The conditions of solvent system were described as follows. Mobile phase included solvents A and B, linear gradients were 0˜15 min (44% A˜49% A), 15˜17.5 min (49% A˜54% A), 17.5˜22.5 min (54% A˜59% A), 22.5˜27.5 min (59% A˜69% A), 27.5˜30 min (69% A˜84% A) and 30˜50 min (84% A˜100% A). Flow rate and column temperature were the same as above.

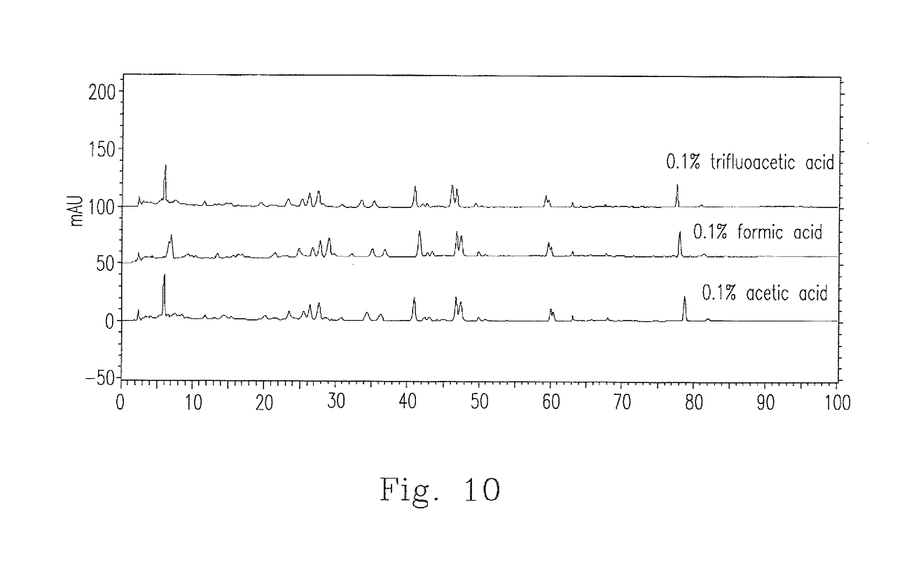

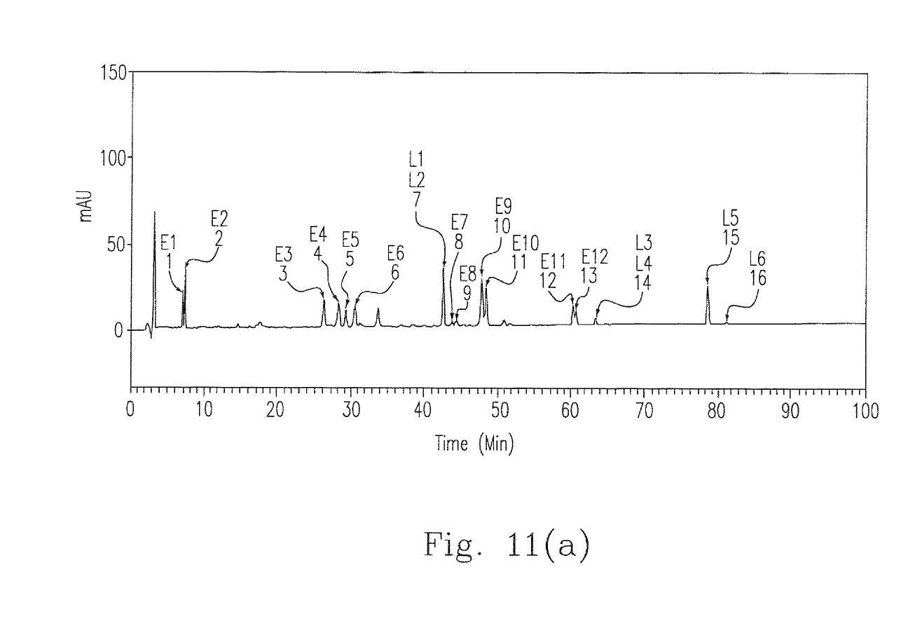

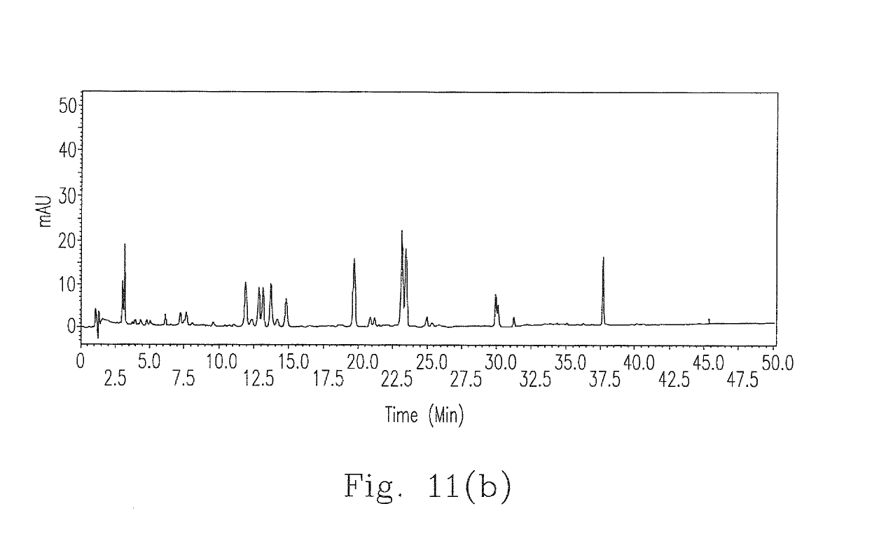

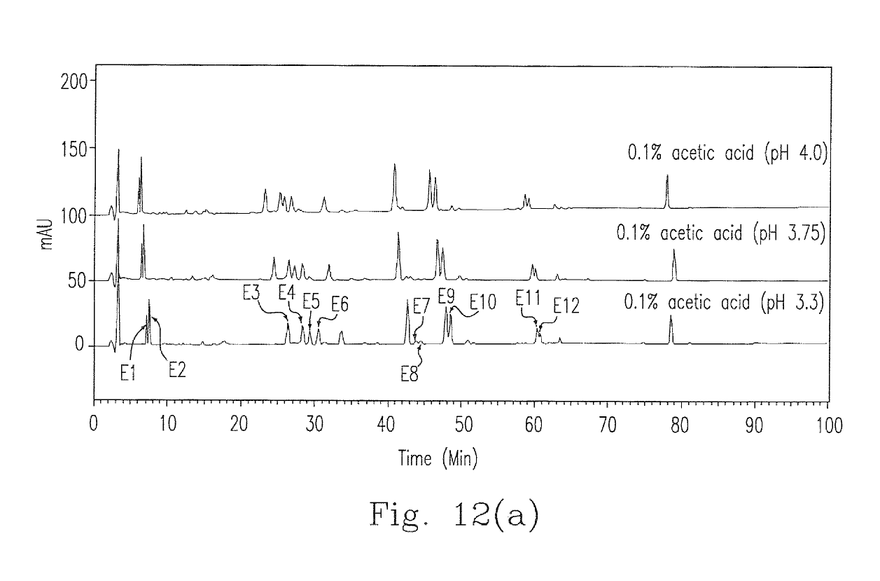

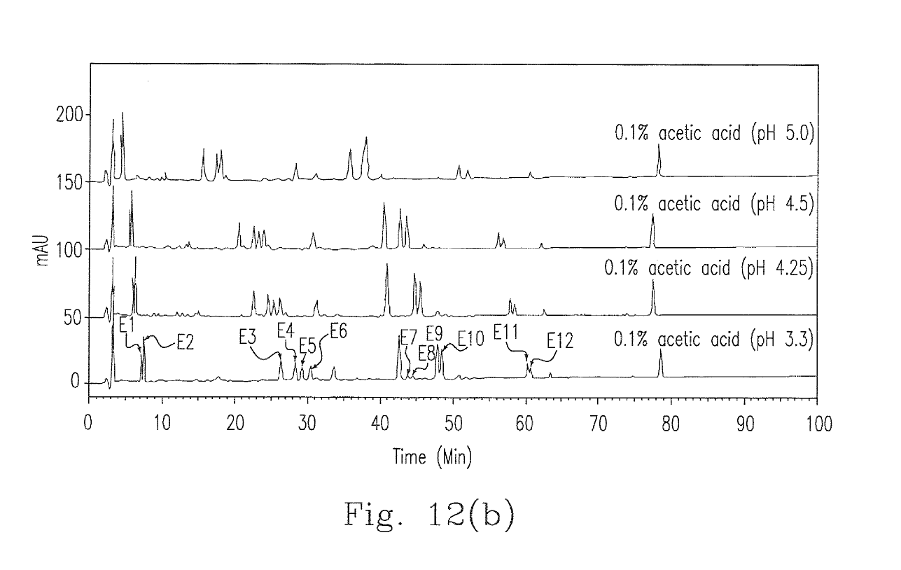

Please refer to FIG. 10, which is the HPLC spectra of EA extract at a wavelength of 254 nm in the different organic acids (0.1% TFA, 0.1% formic acid and 0.1% acetic acid) as the mobile phase. The results indicated that the more stable baseline and resolution were obtained when 0.1% acetic acid (pH 3.30) was the supplemented organic acid in the water mobile phase. Thus, 0.1% acetic acid was chosen to be the organic acid supplemented in the water mobile phase for the analytic conditions, and the compounds represented in each peaks in HPLC spectra were referred to FIGS. 11(a) and 11(b).

Optimization the mobile phase condition in HPLC analysis. However, although the more stable chromatographic baseline could be obtained at the analytic condition that 0.1% acetic acid was contained in the mobile phase, ergostane triterpenoid stereoisomeric mixtures could not be completely isolated. Thus, the acidity coefficient of each ergostane and lanostane triterpenoid compounds were calculated using the online chemical algorithm software “SPARC (Sparc Performs Automated Reasoning in Chemistry)”. Please refer to Table 9, the acidity coefficients of these two types of acidic compounds were ranged in 4.30˜4.60. Next, ammonium acetate (10 mM) then was added, and pH value of water mobile phase was adjusted with pH meter. Five solutions with different pH values were prepared, and the respective pH value was 3.75, 4.00, 4.25, 4.50 and 5.00. HPLC analyses for these five solutions and the original solution (0.1% acetic acid, pH 3.30) were compared. The conditions for HPLC were listed as follows. HPLC apparatus was Shimadzu LC-10AT, detector was Shimadzu SPD-M10A photodiode array detector, auto-sampler was Shimadzu SIL-20A prominence auto sampler, and HPLC column was Cosmosil 5C-18-MS 250×4.6 mm Solvents A and B in mobile phase respectively were CH₃CN and pure water, and 0.1% acetic acid was added to mix with 10 mM ammonium acetate. pH values were adjusted to 3.75, 4.00, 4.25, 4.50 and 5.00. Flow rate was 1 ml/min, the temperature of column was room temperature, and detection wavelength was UV 254 nm. The conditions of solvent system were described as follows. Mobile phase included solvents A and B, the linear gradients were 0˜30 min (45% A˜50% A), 30˜35 min (50% A˜55% A), 35˜45 min (55% A˜60% A), 45˜55 min (60% A˜70% A), 55˜60 min (70% A˜85% A) and 60˜100 min (85% A˜100% A). Flow rate and column temperature were the same as above.

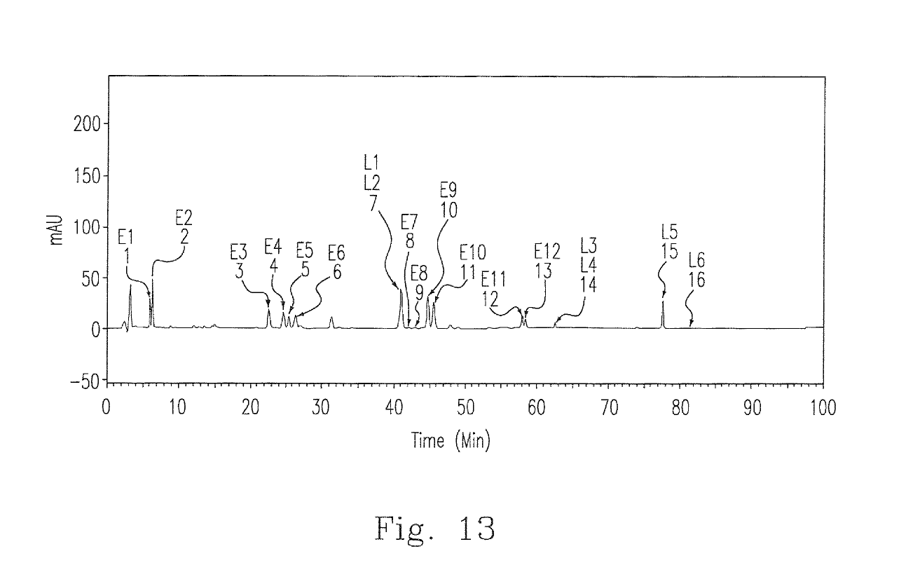

Please refer to FIGS. 12(a) and 12(b), which are the HPLC results of EA extract at a wavelength of 254 nm at the water mobile phase (0.1% acetic acid mixed with 10 mM ammonium acetate) with different pH values. The results revealed that ergostane triterpenoid stereoisomers (compounds E1 to E12) had better resolution and isolation at pH 4.25˜4.50. Thus, it could be determined that the optimal isolation effect could be achieved in the chromatographic spectrum when pH value of mobile phase approached or equaled to the average acidity coefficient of the analytic sample. Please refer to FIG. 13, it could be known from the above experiment that the optimal HPLC conditions for detecting ergostane triterpenoid stereoisomers of fruiting body of AC needed to maintain pH value of mobile phase at 4.25.

Another major component is lanostane triterpenoid compound. Compounds L1 and L2 are similar in structure, compounds L3 and L4 are similar in structure, and compounds L5 and L6 are similar in structure. The structural differences lies in two sets of double bond (at C7-C8 and C9-C11) and one set of double bond (at C8-C9). Although peaks of compounds L1 and L2 are overlapped and peaks of compounds L3 and L4 are overlapped at the eluting gradient condition of this experiment, molecular weights (mw) of compounds L1 and L2 and mw of compounds L3 and L4 are not identical. The qualitative and quantitative determinations of lanostane triterpenoid compounds can be performed by using HPLC-mass spectrometry (MS, e.g. triple quadrupole mass spectrometry) at the above HPLC conditions according to the property of different mw of compounds. The compounds represented in each peaks at the optimal analytic conditions in HPLC spectra were referred to FIG. 13.

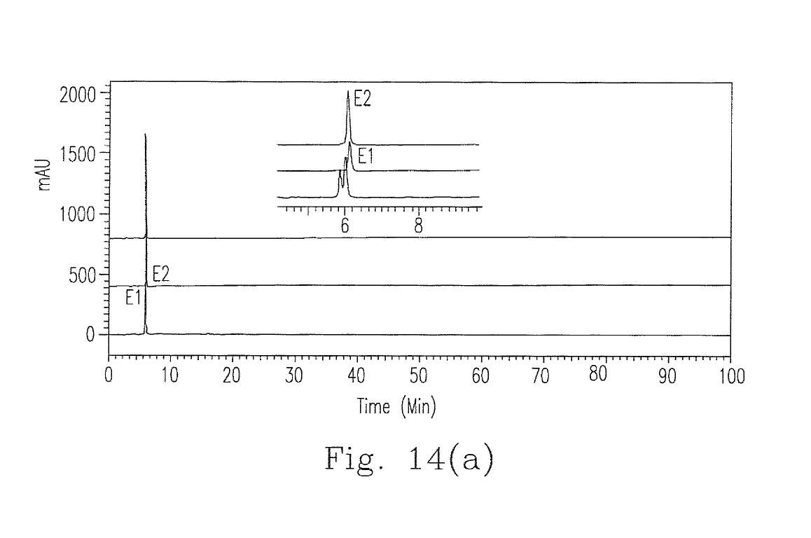

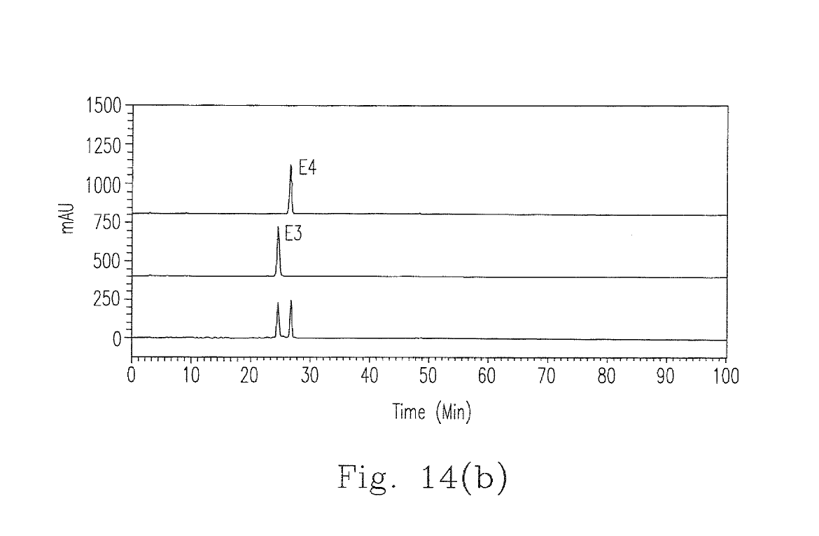

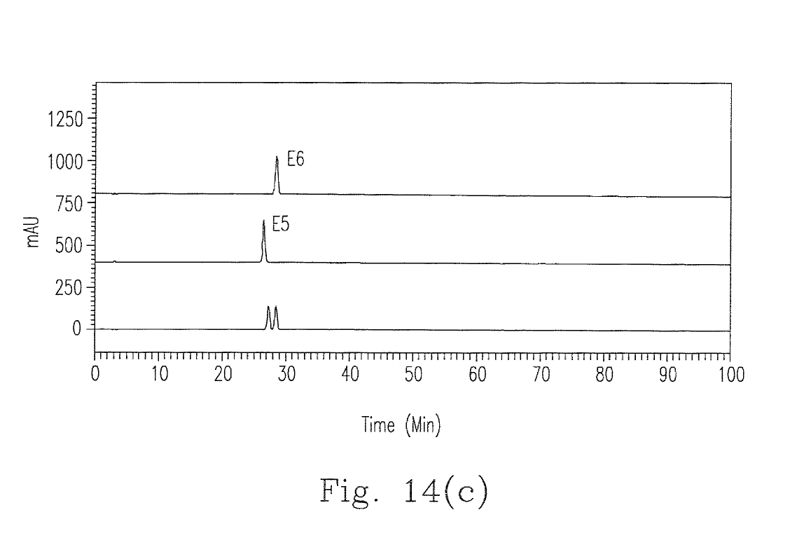

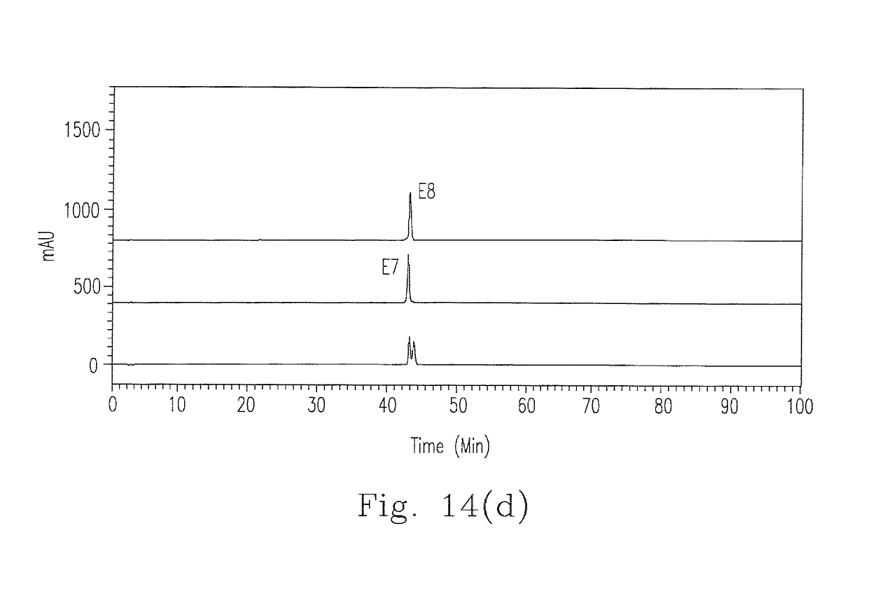

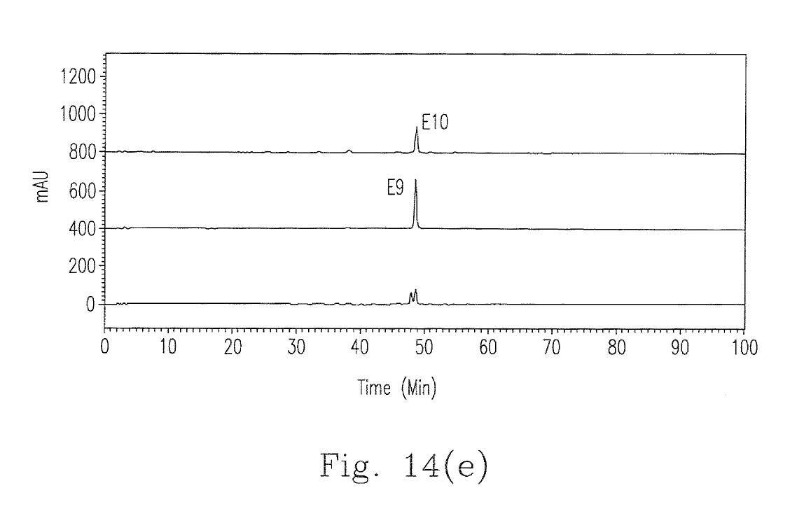

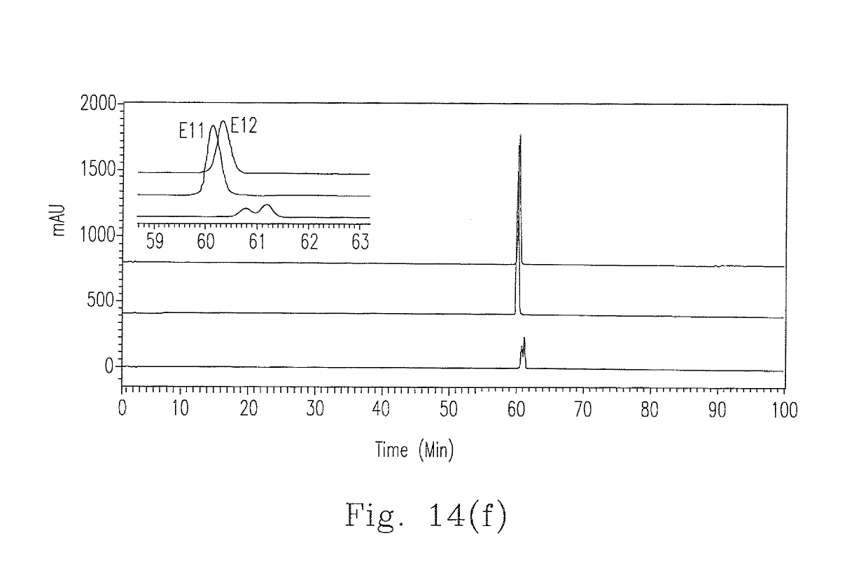

Ergostane triterpenoid stereoisomeric pure compounds E1 to E12 obtained in Experiment 4 and their stereoisomeric mixtures (antcin K, antcin C, zhankuic acid C, zhankuic acid B, zhankuic acid A and antcin A) before purification and isolation were analyzed with HPLC. Please refer to FIGS. 14(a) to 14(f), it was shown on the chromatographic spectra that purities of compounds E1 to E12 achieved more than 95% at the optimal HPLC conditions. In the isolation procedure of stereoisomeric mixtures of ergostane triterpenoids with asymmetrical centers in Experiment 4, solvents A and B used in mobile phase were CH₃CN and water (containing 0.05% acetic acid) respectively, and 0.05% acetic acid was added in its water phase solvent to reach pH 3.53. It was again proved in the above experiment that the optimal isolation effect could be achieved using HPLC when pH value of mobile phase approached or equaled to the average acidity coefficient of the analytic sample, and thus the experiment provided a method for isolating and analyzing ergostane triterpenoid stereoisomeric pure compounds of fruiting body of AC.

### Experiment 8

### NMR Spectrum Analysis

It was known from the above experiment that the major component of EA extract was triterpenoid compound which further was divided as two groups, ergostane and lanostane. Furthermore, the absolute content analyses of the total ergostane triterpenoid compounds and the total lanostane triterpenoid compounds were performed using NMR spectrum analysis method.

The detection experiment procedure was described as follows. First, the appropriate deuterium solvent was selected, and then the standards for these two groups of compounds were respectively chosen to prepare the calibration standard curves with different concentrations. The certain amount of internal standard was added in the standard that was examined, and the integral area ratio of the characteristic signal of the respective standard to target signal of the internal standard was calculated. This integral value and concentration was plotted using linear regression, and thus the calibration curves for standards of two groups of compounds were obtained. Next, EA extract with specific concentration was prepared, and the equal amount of deuterium solvent and the internal standard were added to proceed NMR spectrum analysis. The characteristic signals of two groups of compounds and the target signals of internal standard were detailedly integrated to calculate the integral ratios, and the absolute contents of two groups of compounds in EA extract were obtained based on the calibration curves.

The quantitative analysis of the total ergostane triterpenoid compounds and the total lanostane triterpenoid compounds in EA extract was proceeded using NMR spectrum analysis method in the present invention. The experimental conditions were listed as follows. The standards of two groups of compounds with different concentrations were prepared, which respectively were zhankuic acid A of ergostane triterpenoid and dehydroeburicoic acid of lanostane triterpenoid. The internal standard (pyrazine, 0.132 mg) was added and dissolved in DMSO-d6 solution (0.6 ml), which was the test solvent for NMR spectrum analysis (CDCl₃ and C₅D₅N also could be chosen, but they had problems such as signal interference and solubility; data not shown). NMR apparatus was Varian UNITY plus 400 MHz spectrometer, the scanning times was 10 (7 min), the spectrum width was 6002.4 Hz, and width for impulse strength was 6.3 μs. Please refer to Tables 10 and 11, the start point and end point for C-28 methylene characteristic signal of the standard for two groups of compounds were further manually selected to calculate peak integral area, and integral area ratio of the peak integral area to the target signal of internal standard pyrazine (δ_{H} 8.66) was calculated. The characteristic proton absorption signal of the standard zhankuic acid A was situated at δ_{H} 4.82 (2H, br d), and that of the standard dehydroeburicoic acid was situated at δ_{H} 4.63 (1H, s) and 4.70 (1H, s). The whole experiment was made in triplicate and the relative standard deviation value (RSD %) was determined. Please refer to Table 12, the integral ratio and concentration further was plotted using linear regression, and calibration curve (standard curve and the determination coefficient of regression analysis) for the standard of two groups of compounds could be obtained and to be the basis of this quantitative analysis method.

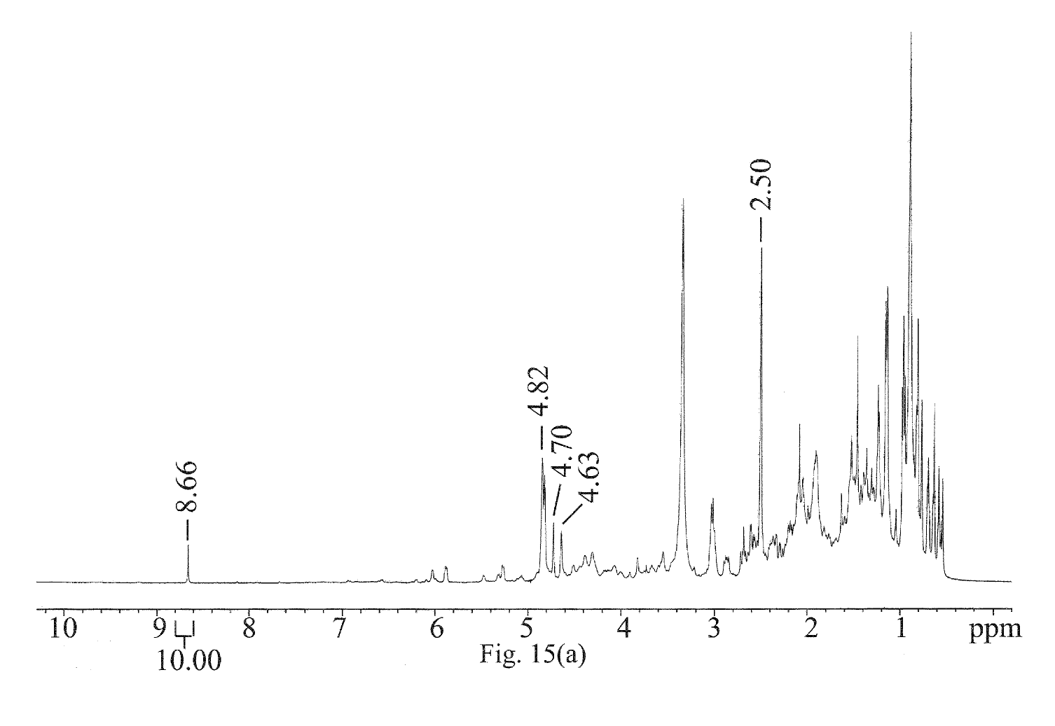

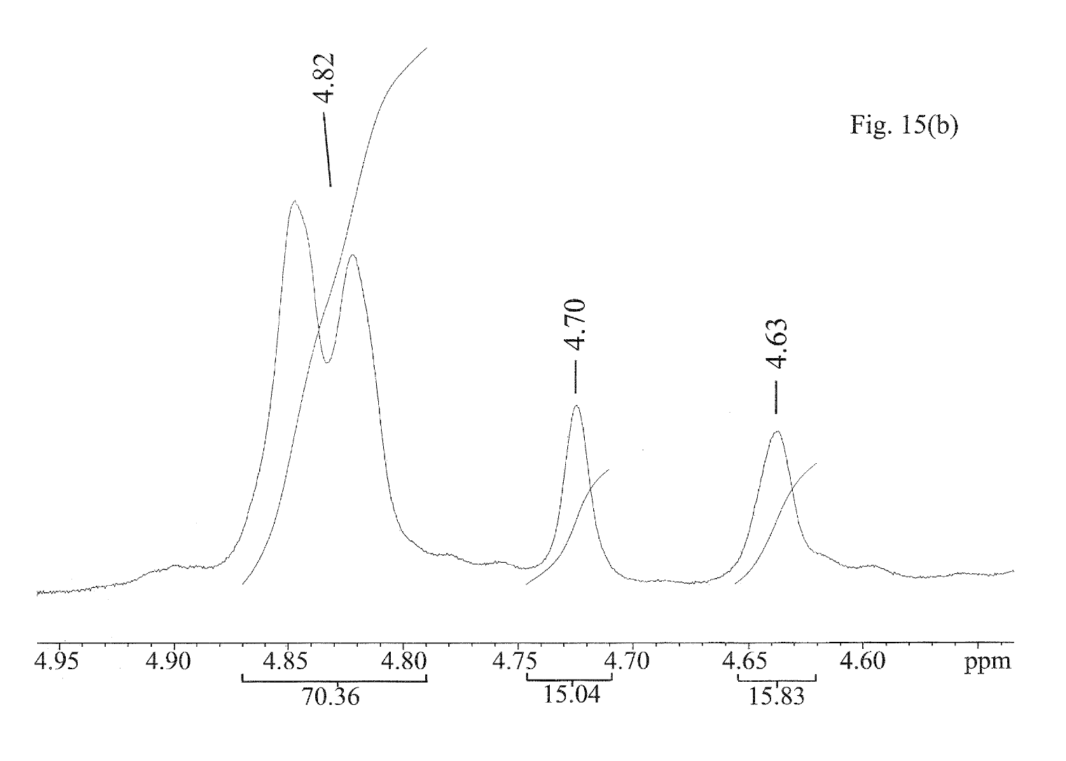

After obtaining the calibration curves for standards of two groups of compounds, EA extract (20.12 mg) was further prepared, the equal amount of DMSO-d6 and internal standard pyrazine were added, and NMR spectrum analysis was proceeded. Please refer to FIG. 15 and Table 13, the C-28 methylene characteristic signals for standards of two groups of compounds in EA extract and the target signals of the internal standard in the NMR spectrum were detailedly integrated to calculate the integral ratios. The whole experiment was made in triplicate and RSD % was determined. The absolute contents for two groups of compounds in EA extract were obtained based on the calibration curves for standards of above obtained two groups of compounds.

It could be known from the results that the absolute content of total ergostane triterpenoid compound was 5.67 mg and that of total lanostane triterpenoid compound was 2.71 mg in 20.12 mg of EA extract. In accordance with the calibration curve for standard of two groups of compound obtained from NMR spectrum and the RSD values within the acceptable range, this method not only is rapid but also has reproducibility.

While the invention has been described in terms of what is presently considered to be the most practical and preferred Embodiments, it is to be understood that the invention needs not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

TABLE 1
¹H and ¹³C NMR data of compounds E1 and E2
(600 and 150 MHz in C₅D₅N, δ in ppm, J in Hz)

| | Compound E1 | | Compound E2 | |
|---|---|---|---|---|
| Position | δ_{H} (J in Hz) | δ_{C} | δ_{H} (J in Hz) | δ_{C} |
| 1 | a 2.110 m | 29.687 | a 2.108 m | 29.687 |
| | b 3.148 dt (13.2, 3.0) | | b 3.149 dt (13.2, 3.6) | |
| 2 | a 1.965 m | 26.786 | a 1.975 m | 26.785 |
| | b 2.771 m | | b 2.778 m | |
| 3 | β 4.092 s | 74.711 | β 4.094 s | 74.711 |
| 4 | | 73.957 | | 73.957 |
| 5 | α 2.202 m | 43.498 | α 2.201 m | 43.498 |
| 6 | a 2.461 m | 30.199 | a 2.466 m | 30.199 |
| | b 2.749 m | | b 2.750 m | |
| 7 | α 4.650 t (8.4) | 70.805 | α 4.651 br t | 70.805 |
| 8 | | 154.299 | | 154.292 |
| 9 | | 143.939 | | 143.939 |
| 10 | | 38.755 | | 38.751 |
| 11 | | 201.504 | | 201.504 |
| 12 | a 2.476 m | 58.817 | a 2.462 m | 58.817 |
| | b 3.000 d (13.2) | | b 3.000 d (13.8) | |
| 13 | | 47.942 | | 47.938 |
| 14 | α 2.666 m | 53.768 | α 2.674 m | 53.772 |
| 15 | a 2.120 m | 25.486 | a 2.128 m | 25.490 |
| | b 2.546 m | | b 2.541 m | |
| 16 | a 1.328 m | 28.298 | a 1.360 m | 28.234 |
| | b 1.965 m | | b 1.952 m | |
| 17 | α 1.441 m | 54.855 | α 1.436 m | 54.877 |
| 18 | 0.929 s | 12.493 | 0.925 s | 12.493 |
| 19 | 2.099 s | 20.956 | 2.098 s | 20.956 |
| 20 | β 1.405 m | 36.242 | β 1.417 m | 36.271 |
| 21 | 0.908 d (6.0) | 18.614 | 0.909 d (6.0) | 18.655 |
| 22 | a 1.328 m | 34.419 | a 1.297 m | 34.509 |
| | b 1.740 m | | b 1.789 m | |
| 23 | a 2.237 m | 31.999 | a 2.238 m | 31.764 |
| | b 2.498 m | | b 2.439 m | |
| 24 | | 150.833 | | 150.707 |
| 25 | 3.485 br q | 47.068 | 3.491 q (7.2) | 47.005 |
| 26 | | 177.842 | | 177.237 |
| 27 | 1.534 d (6.6) | 17.165 | 1.530 d (7.2) | 17.255 |
| 28 | a 5.076 s | 110.003 | a 5.083 s | 110.186 |
| | b 5.234 s | | b 5.256 s | |
| 29 | 1.763 s | 28.059 | 1.765 s | 28.059 |

TABLE 2
¹H and ¹³C NMR data of compounds E3 and E4
(600 and 150 MHz in C₅D₅N, δ in ppm, J in Hz)

| | Compound E3 | | Compound E4 | |
|---|---|---|---|---|
| Position | δ_{H} (J in Hz) | δ_{C} | δ_{H} (J in Hz) | δ_{C} |
| 1 | a 1.416 m | 36.163 | a 1.436 m | 36.160 |
| | b 3.232 qd (6.0, 2.4) | | b 3.231 qd (6.6, 2.4) | |
| 2 | a 2.390 m | 38.123 | a 2.389 m | 38.121 |
| | b 2.548 m | | b 2.537 m | |
| 3 | | 211.368 | | 211.359 |
| 4 | β 2.374 m | 44.069 | β 2.373 m | 44.074 |
| 5 | α 1.416 m | 48.629 | α 1.414 m | 48.634 |
| 6 | a 1.823 m | 33.504 | a 1.822 m | 33.509 |
| | b 2.374 m | | b 2.373 m | |
| 7 | α 4.524 t (7.8) | 69.311 | α 4.527 td (8.4, 1.2) | 69.319 |
| 8 | | 155.860 | | 155.856 |
| 9 | | 140.862 | | 140.873 |
| 10 | | 37.391 | | 37.396 |
| 11 | | 201.318 | | 201.317 |
| 12 | a 2.473 d (13.8) | 58.454 | a 2.477 d (13.8) | 58.470 |
| | b 3.000 d (13.8) | | b 3.000 d (13.8) | |
| 13 | | 47.882 | | 47.891 |
| 14 | α 2.755 ddd | 53.577 | a 2.763 ddd | 53.597 |
| | (12.0, 6.6, 1.2) | | (12.6, 7.2, 1.8) | |
| 15 | a 2.113 m | 25.362 | a 2.116 m | 25.371 |
| | b 2.548 m | | b 2.537 m | |
| 16 | a 1.336 m | 28.227 | a 1.337 m | 28.183 |
| | b 1.947 m | | b 1.924 m | |
| 17 | α 1.416 m | 54.645 | α 1.424 m | 54.699 |
| 18 | 0.893 s | 12.470 | 0.891 s | 12.480 |
| 19 | 1.604 s | 17.643 | 1.602 s | 17.648 |
| 20 | β 1.336 m | 36.114 | β 1.335 m | 36.160 |
| 21 | 0.911 d (6.0) | 18.603 | 0.913 d (6.0) | 18.657 |
| 22 | a 1.354 m | 34.359 | a 1.313 m | 34.472 |
| | b 1.737 td (12.0, 5.4) | | b 1.775 m | |
| 23 | a 2.236 m | 31.860 | a 2.235 m | 31.675 |
| | b 2.484 m | | b 2.431 m | |
| 24 | | 150.601 | | 150.616 |
| 25 | 3.487 br q (6.6) | 46.720 | 3.483 q (6.6) | 46.923 |
| 26 | | 177.371 | | 177.154 |
| 27 | 1.530 d (6.6) | 17.086 | 1.522 d (7.2) | 17.234 |
| 28 | a 5.089 s | 110.280 | a 5.085 s | 110.302 |
| | b 5.242 s | | b 5.256 s | |
| 29 | 1.132 d (6.6) | 11.888 | 1.132 d (6.6) | 11.893 |

TABLE 3
¹H and ¹³C NMR data of compounds E5 and E6
(600 and 150 MHz in C₅D₅N, δ in ppm, J in Hz)

| | Compound E5 | | Compound E6 | |
|---|---|---|---|---|
| Position | δ_{H} (J in Hz) | δ_{C} | δ_{H} (J in Hz) | δ_{C} |
| 1 | a 1.957 m | 28.583 | a 1.946 m | 28.575 |
| | b 2.737 dt (12.0, 3.6) | | b 2.734 dt (13.2, 3.0) | |
| 2 | a 1.855 m | 30.155 | a 1.857 m | 30.148 |
| | b 1.855 m | | b 1.857 m | |
| 3 | β 3.877 s | 69.308 | β 3.874 br s | 69.301 |
| 4 | β 1.692 m | 35.324 | β 1.699 m | 35.316 |
| 5 | α 2.596 m | 41.605 | α 2.592 m | 41.602 |
| 6 | a 2.448 m | 38.685 | a 2.448 m | 38.677 |
| | b 2.611 m | | b 2.613 m | |
| 7 | | 202.004 | | 201.993 |
| 8 | | 144.406 | | 144.395 |
| 9 | | 153.160 | | 153.145 |
| 10 | | 38.980 | | 38.976 |
| 11 | | 203.938 | | 203.927 |
| 12 | β 4.505 s | 80.956 | β 4.500 s | 80.941 |
| 13 | | 50.240 | | 50.225 |
| 14 | α 3.567 dd (13.2, 7.8) | 42.707 | α 3.559 dd (13.2, 7.8) | 42.692 |
| 15 | a 1.674 m | 24.617 | a 1.677 m | 24.598 |
| | b 2.858 m | | b 2.854 m | |
| 16 | a 1.327 m | 27.351 | a 1.315 m | 27.377 |
| | b 1.978 m | | b 1.982 m | |
| 17 | α 2.302 m | 46.079 | α 2.300 m | 46.027 |
| 18 | 0.821 s | 11.815 | 0.819 s | 11.796 |
| 19 | 1.547 s | 16.457 | 1.547 s | 16.449 |
| 20 | β 1.490 m | 35.974 | β 1.478 m | 35.891 |
| 21 | 1.077 d (6.6) | 18.141 | 1.075 d (6.6) | 18.070 |
| 22 | a 1.346 m | 34.640 | a 1.389 m | 34.491 |
| | b 1.806 m | | b 1.767 m | |
| 23 | a 2.236 m | 31.843 | a 2.233 m | 32.052 |
| | b 2.423 m | | b 2.474 m | |
| 24 | | 150.830 | | 150.490 |
| 25 | 3.457 q (7.2) | 47.095 | 3.452 q (7.2) | 46.602 |
| 26 | | 177.479 | | 177.113 |
| 27 | 1.496 d (7.2) | 17.290 | 1.504 d (6.6) | 17.058 |
| 28 | a 5.059 s | 110.145 | a 5.073 s | 110.291 |
| | b 5.234 s | | b 5.226 s | |
| 29 | 1.052 d (6.6) | 16.394 | 1.050 d (7.2) | 16.393 |

TABLE 4
¹H and ¹³C NMR data of compounds E9 and E10
(600 and 150 MHz in C₅D₅N, δ in ppm, J in Hz)

| | Compound E9 | | Compound E10 | |
|---|---|---|---|---|
| Position | δ_{H} (J in Hz) | δ_{C} | δ_{H} (J in Hz) | δ_{C} |
| 1 | a 1.437 m | 34.947 | a 1.422 m | 34.936 |
| | b 3.178 qd (6.6, 3.0) | | b 3.178 qd (6.6, 2.4) | |
| 2 | a 2.406 m | 37.771 | a 2.406 m | 37.767 |
| | b 2.588 m | | b 2.570 m | |
| 3 | | 209.898 | | 209.909 |
| 4 | β 2.464 m | 43.925 | β 2.458 m | 43.918 |
| 5 | α 1.886 m | 48.914 | α 1.880 m | 48.896 |
| 6 | a 2.584 m | 39.208 | a 2.570 m | 39.201 |
| | b 2.584 m | | b 2.570 m | |
| 7 | | 200.778 | | 200.789 |
| 8 | | 145.504 | | 145.504 |
| 9 | | 151.957 | | 151.953 |
| 10 | | 38.630 | | 38.618 |
| 11 | | 202.679 | | 202.701 |
| 12 | a 2.503 m | 57.474 | a 2.503 m | 57.470 |
| | b 3.019 d (13.8) | | b 3.018 d (13.8) | |
| 13 | | 47.238 | | 47.234 |
| 14 | α 2.742 m | 49.471 | α 2.745 m | 49.463 |
| 15 | a 1.547 m | 25.297 | a 1.552 m | 25.301 |
| | b 2.753 m | | b 2.734 m | |
| 16 | a 1.240 m | 28.027 | a 1.242 m | 27.979 |
| | b 1.915 m | | b 1.906 m | |
| 17 | α 1.390 m | 54.001 | α 1.382 m | 54.016 |
| 18 | 0.707 s | 12.092 | 0.703 s | 12.092 |
| 19 | 1.611 s | 16.249 | 1.609 s | 16.241 |
| 20 | β 1.381 m | 35.881 | β 1.390 m | 35.959 |
| 21 | 0.895 d (5.4) | 18.549 | 0.892 d (6.0) | 18.601 |
| 22 | a 1.314 m | 34.253 | a 1.272 m | 34.383 |
| | b 1.697 td (11.4, 5.4) | | b 1.738 td (12.0, 3.6) | |
| 23 | a 2.211 m | 31.855 | a 2.223 m | 31.657 |
| | b 2.448 m | | b 2.406 m | |
| 24 | | 150.642 | | 151.116 |
| 25 | 3.464 br q (7.2) | 47.006 | 3.480 br q (6.6) | 47.518 |
| 26 | | 177.770 | | 178.124 |
| 27 | 1.524 d (7.2) | 17.138 | 1.529 d (6.6) | 17.425 |
| 28 | a 5.069 s | 110.127 | a 5.060 s | 109.888 |
| | b 5.231 s | | b 5.248 s | |
| 29 | 1.039 d (6.6) | 11.558 | 1.039 d (6.6) | 11.551 |

TABLE 5
¹H NMR data of characteristics of compounds E3-1RAT, E3-1SAT,
E4-1RAT, and E4-1SAT (600 MHz in C₅D₅N, δ in ppm, J in Hz)

| | Compound E3 | | Compound E4 | |
|---|---|---|---|---|
| Position | 1RAT | 1SAT | 1RAT | 1SAT |
| 18 | 0.814 s | 0.891 s | 0.912 s | 0.813 s |
| 19 | 1.613 s | 1.624 s | 1.621 s | 1.618 s |
| 21 | 0.556 d (6.6) | 0.807 d (6.0) | 0.839 d (6.6) | 0.574 d (6.0) |
| 27 | 1.388 d (7.2) | 1.356 d (7.2) | 1.334 d (7.2) | 1.385 d (7.2) |
| 28 | a 4.918 s | a 5.127 s | a 5.121 s | a 4.911 s |
| | b 5.045 s | b 5.173 s | b 5.176 s | b 5.007 s |
| 29 | 1.142 d (6.6) | 1.147 d (6.0) | 1.141 d (6.6) | 1.146 d (6.6) |

TABLE 6
¹H NMR data of characteristics of compounds E5-1RAT, E5-1SAT,
E6-1RAT, and E6-1SAT (600 MHz in C₅D₅N, δ in ppm, Jin Hz)

| | Compound E5 | | Compound E6 | |
|---|---|---|---|---|
| Position | 1RAT | 1SAT | 1RAT | 1SAT |
| 18 | 0.844 s | 0.739 s | 0.751 s | 0.821 s |
| 19 | 1.565 s | 1.563 s | 1.557 s | 1.567 s |
| 21 | 1.029 d (6.6) | 0.800 d (6.6) | 0.771 d (6.6) | 1.016 d (6.6) |
| 27 | 1.302 d (7.2) | 1.355 d (7.2) | 1.365 d (7.2) | 1.329 d (7.2) |
| 28 | a 5.108 s | a 4.877 s | a 4.895 s | a 5.116 s |
| | b 5.156 s | b 4.980 s | b 5.007 s | b 5.157 s |
| 29 | 1.059 d (7.2) | 1.071 d (6.6) | 1.062 d (7.2) | 1.067 d (6.6) |

TABLE 7
¹H NMR data of characteristics of compounds E9-1RAT, E9-1SAT,
E10-1RAT, and E10-1SAT (600 MHz in C₅D₅N, δ in ppm, J in Hz)

| | Compound E9 | | Compound E10 | |
|---|---|---|---|---|
| Position | 1RAT | 1SAT | 1RAT | 1SAT |
| 18 | 0.611 s | 0.690 s | 0.713 s | 0.607 s |
| 19 | 1.617 s | 1.626 s | 1.626 s | 1.619 s |
| 21 | 0.521 d (6.0) | 0.787 d (6.0) | 0.815 d (6.0) | 0.560 d (6.0) |
| 27 | 1.389 d (7.2) | 1.360 d (6.6) | 1.338 d (7.2) | 1.386 d (7.2) |
| 28 | a 4.912 s | a 5.121 s | a 5.113 s | a 4.906 s |
| | b 5.051 s | b 5.175 s | b 5.177 s | b 5.023 s |
| 29 | 1.049 d (6.6) | 1.053 d (6.6) | 1.046 d (7.2) | 1.054 d (6.6) |

TABLE 8
Cytotoxicity test of the major ergostane triterpenoid compounds and
their stereoisomeric pure compounds

| | IC₅₀ (μg/ml)/Cell line | | |
|---|---|---|---|
| Compound | CCRF-CEM^{a} | Molt 4^{b} | HL 60^{c} |
| E1 | >80 | >80 | >80 |
| E2 | >80 | >80 | >80 |
| E3 | 30.681 ± 5.30 | 77.04 ± 2.78 | >80 |
| E4 | 27.94 ± 6.44 | 54.28 ± 1.96 | >80 |
| E5 | >80 | >80 | >80 |
| E6 | >80 | >80 | >80 |
| E9 | 21.99 ± 7.91 | 42.16 ± 2.33 | 54.67 ± 8.14 |
| El0 | 22.90 ± 7.60 | 16.44 ± 3.77 | 23.32 ± 1.60 |
| Zhankuic acid A | 47.04 ± 6.191 | 53.23 ± 3.88 | 69.98 ± 18.98 |
| Zhankuic acid C | >80 | >80 | >80 |
| Antcin C | 28.82 ± 6.79 | 55.02 ± 3.34 | 47.02 ± 4.45 |
| Antcin K | >80 | >80 | >80 |

^{a}and ^{b}human acute lymphoblastic leukemia cells
^{c}human promyelocytic leukemia cells

TABLE 9
Acidity coefficient (pKa) of ergostane and lanostane compounds calculated by the online chemical
algorithm software “SPARC Performs Automated Reasoning in Chemistry”

| | | | Acidity |
|---|---|---|---|
| | Com- | | coefficient |
| Index | pound | Structure | (pKa) |
| 1 | E1 | C[C@]34CC(═O)C1═C([C@]([H])(C[C@@]2([H])[C@@](C)(O[H])[C@@]([H])(CC[C@] | 4.45 |
| | | 12C)O[H])O[H])[C@]3([H])CC[C@]4([H])[C@@](C)([H])CCC(═C)[C@@](C)([H])C(═O) | |
| | | O[H] | |
| 2 | E2 | C[C@]34CC(═O)C1═C([C@]([H](C[C@@]2([H])[C@@](C)(O[H])[C@@]([H])(CC[C@] | 4.45 |
| | | 12C)O[H])O[H])[C@]3([H])CC[C@]4([H])[C@@](C)([H])CCC(═C)[C@](C)([H])C(═O)O[H] | |
| 3 | E3 | O═C2CC[C@]1(C)C4═C([C@]([H])(C[C@@]1([H])[C@@]2(C)[H])O[H])[C@]3([H])CC[ | 4.45 |
| | | C@@]([H])([C@@]3(C)CC4═O)[C@@](C)([H])CCC(═C)[C@@](C)([H])C(═O)O[H] | |
| 4 | E4 | O═C2CC[C@]1(C)C4═C([C@]([H])(C[C@@]1([H])[C@@]2(C)[H])O[H])[C@]3([H])CC[ | 4.45 |
| | | C@@]([H])([C@@]3(C)CC4═O)[C@@](C)([H])CCC(═C)[C@](C)([H])C(═O)O[H] | |
| 5 | E5 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@@](C)([H])C(═O)O[H])O[H] | |
| 6 | E6 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@](C)([H])C(═O)O[H])O[H] | |
| 7 | E7 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@@](C)([H])C(═O)O[H])O[H] | |
| 8 | E8 | O═C2C[C@@]1([H])[C@@](C)([H])[C@@]([H])(CC[C@]1(C)C4═C2[C@]3([H])CC[C@ | 4.45 |
| | | @]([H])([C@@]3(C)CC4═O)[C@@](C)([H])CCC(═C)[C@](C)([H])C(═O)O[H])O[H] | |
| 9 | E9 | O═C2CC[C@]1(C)C4═C(C(═O)C[C@@]1([H])[C@@]2(C)[H])[C@]3([H])CC[C@@]([H]) | 4.45 |
| | | ([C@@]3(C)CC4═O)[C@@](C)([H])CCC(═C)[C@@](C)([H])C(═O)O[H] | |
| 10 | E10 | O═C2CC[C@]1(C)C4═C(C(═O)C[C@@]1([H])[C@@]2(C)[H])[C@]3([H])CC[C@@]([H]) | 4.45 |
| | | ([C@@]3(C)CC4═O)[C@@](C)([H])CCC(═C)[C@](C)([H])C(═O)O[H] | |
| 11 | E11 | O═C2CC[C@]1(C)C4═C(CC[C@@]1([H])[C@@]2(C)[H])[C@]3([H])CC[C@@]([H])([C@ | 4.45 |
| | | @]3(C)CC4═O)[C@@](C)([H])CCC(═C)[C@@](C)([H])C(═O)O[H] | |
| 12 | E12 | O═C2CC[C@]1(C)C4═C(CC[C@@]1([H])[C@@]2(C)[H])[C@]3([H])CC[C@@]([H])([C@ | 4.45 |
| | | @]3(C)CC4═O)[C@@](C)([H])CCC(═C)[C@](C)([H])C(═O)O[H] | |
| 13 | L1 | C[C@]34CC═C1C(═CC[C@@]2([H])2([H])C(C)(C)[C@]([H])(CC[C@]12C)O[H])[C@]3(C) | 4.44 |
| | | [C@]([H])(C[C@]4([H])[C@@]([H])(CCC(═C)C(C)(C)[H])C(═O)O[H])O[H] | |
| 14 | L2 | C[C@]34CCC1═C(CC[C@@]2([H])C(C)(C)[C@]([H])(CC[C@]12C)O[H])[C@]3(C)[C@]([ | 4.49 |
| | | H])(C[C@]4([H])[C@@]([H])(CCC(═C)C(C)(C)[H])C(═O)O[H])O[H] | |
| 15 | L3 | C[C@]34CC═C1C(═CC[C@@]2([H])C(C)(C)[C@]([H])(CC[C@]12C)O[H])[C@]3(C)[C@]([ | 4.3 |
| | | H])(C[C@]4([H])[C@@]([H])(CCC(═C)C(C)(C)[H])C(═O)O[H])OC(═O)C | |
| 16 | L4 | C[C@]34CCC1═C(CC[C@@]2([H])C(C)(C)[C@]([H])(CC[C@]12C)O[H])[C@]3(C)[C@]([ | 4.36 |
| | | H])(C[C@]4([H])[C@@]([H])(CCC(═C)C(C)(C)[H])C(═O)O[H])OC(═O)C | |
| 17 | L5 | C[C@]34CC═C1C(═CC[C@@]2([H])C(C)(C)[C@]([H])(CC[C@[12C)O[H])[C@]3(C)CC[C | 4.54 |
| | | @]4([H])[C@@]([H])(CCC(═C)C(C)(C)[H])C(═O)O[H] | |
| 18 | L6 | CC(C)([H])C(═C)CC[C@@]([H])(C(═O)O[H])[C@@]4([H])CC[C@@]3(C)C═2CC[C@@]1( | 4.59 |
| | | [H])C(C)(C)[C@]([H])(CC[C@]1(C)C═2CC[C@]34C)O[H] | |

TABLE 10
Integral area ratio of C-28 methylene signal of zhankuic acid A to the
internal standard and its relative standard deviation

| Weight | Integral area ratio | | | | |
|---|---|---|---|---|---|
| (mg) | 1 | 2 | 3 | Average | RSD % |
| 2.020 | 28.07 | 28.47 | 28.58 | 28.37 | 0.95 |
| 3.030 | 38.61 | 38.68 | 38.75 | 38.68 | 0.18 |
| 4.000 | 49.31 | 48.74 | 49.95 | 49.33 | 1.23 |
| 5.040 | 63.82 | 63.99 | 64 | 63.94 | 0.16 |
| 6.060 | 75.8 | 75.39 | 75.11 | 75.43 | 0.46 |

TABLE 11
Integral area ratio of C-28 methylene signal of dehydroeburicoic acid
to the internal standard and its relative standard deviation

| Weight | Integral ratio | | | | |
|---|---|---|---|---|---|
| (mg) | 1 | 2 | 3 | Average | RSD % |
| 1.150 | 13.54 | 13.4 | 13.49 | 13.48 | 0.53 |
| 2.100 | 24.44 | 24.07 | 24.68 | 24.40 | 1.26 |
| 3.050 | 35.39 | 34.91 | 34.56 | 34.95 | 1.19 |
| 4.040 | 46.58 | 46.01 | 46.37 | 46.32 | 0.62 |
| 5.010 | 55.77 | 55.87 | 55.6 | 55.75 | 0.24 |

TABLE 12
Standard curves of zhankuic acid A and dehydroeburicoic acid

| Compound | Standard curve | Determination coefficient |
|---|---|---|
| Zhankuic acid A | Y = 11.8 X + 3.5 | 0.997 |
| Dehydroeburicoic acid | Y = 11.0 X + 1.1 | 0.999 |

TABLE 13
Signal integral ratios of C-28 methylene signals of zhankuic acid A
(20.12 mg) and dehydroeburicoic acid to the internal standard pyrazine

| | Integral ratio | | | | |
|---|---|---|---|---|---|
| Compound | 1 | 2 | 3 | Average | RSD % |
| Zhankuic acid A | 70.36 | 70.41 | 70.15 | 70.31 | 0.16 |
| Dehydroeburicoic acid | 30.87 | 30.88 | 31.01 | 30.92 | 0.21 |

What is claimed is:

1. A pharmaceutical composition comprising at least one ergostane triterpenoid compound being represented by one selected from a group consisting of the following formulas I, II, III, IV, V, VI, VIII, IX and a combination thereof:

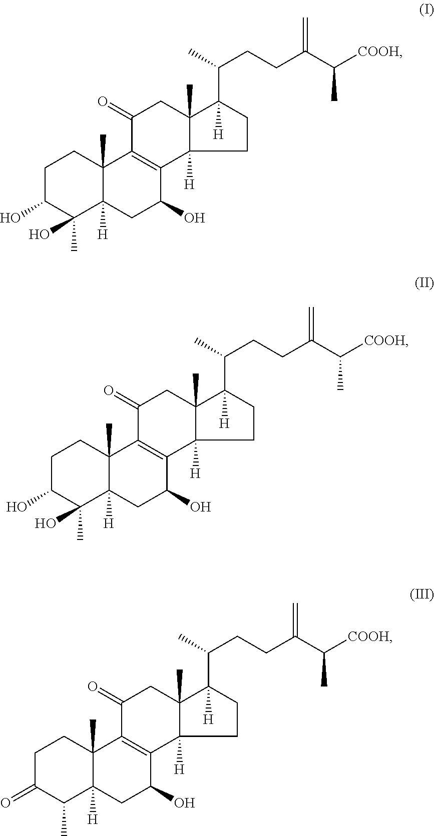

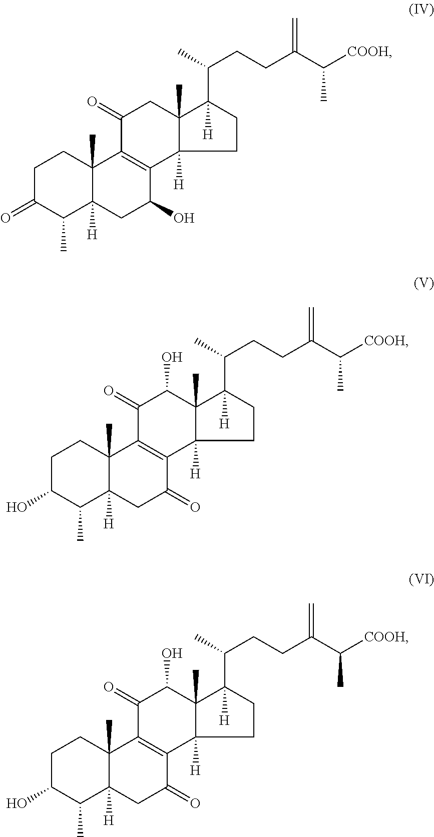

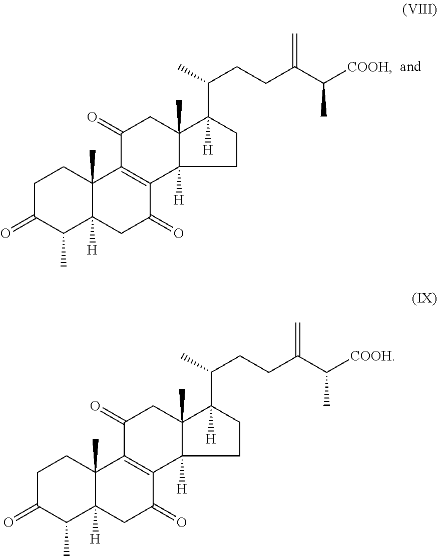

2. The pharmaceutical composition according to claim 1, wherein the at least one ergostane triterpenoid compound has a cytotoxicity to leukemia cells.

3. A method for preparing an ergostane triterpenoid compound, comprising steps of:
providing an ethyl acetate extract of a fruiting body of an Antrodia cinnamomea; and
chromatographing the ethyl acetate extract to obtain the ergostane triterpenoid compound being one selected from a group consisting of a 3α,4β,7β-trihydroxy-4α-methylergosta-8,24(28)-dien-11-on-25S-26-oic acid, a 3α,4β,7β-trihydroxy-4α-methylergosta-8,24(28)-dien-11-on-25R-26-oic acid, a 7β-hydroxy-4α-methylergosta-8,24(28)-dien-3,11-dion-25S-26-oic acid, a 7β-hydroxy-4α-methylergosta-8,24(28)-dien-3,11-dion-25R-26-oic acid, a 3α,12α-dihydroxy-4α-methylergosta-8,24(28)-dien-7,11-dion-25R-26-oic acid, a 3α,12α-dihydroxy-4α-methylergosta-8,24(28)-dien-7,11-dion-25S-26-oic acid, a 4α-methylergosta-8,24(28)-dien-3,7,11-trion-25S-26-oic acid, a 4α-methylergosta-8,24(28)-dien-3,7,11-trion-25R-26-oic acid and a combination thereof.

4. The method according to claim 3, wherein the ethyl acetate extract is obtained by sequentially extracting the fruiting body of A. cinnamomea with an ethanol solution, an n-hexane solution and an ethyl acetate solution.

5. The method according to claim 3 further comprising a step of chromatographing the ethyl acetate extract to obtain a lanostane triterpenoid compound.

6. The method according to claim 5, wherein the lanostane triterpenoid compound is one selected from a group consisting of a dehydrosulphurenic acid, a sulphurenic acid, a 15α-acetyl-dehydrosulphurenic acid, a versisponic acid D, a dehydroeburicoic acid, an eburicoic acid and a combination thereof.

7. The method according to claim 3, wherein the ergostane triterpenoid compound comprises a stereoisomer, and the method further comprises a step of isolating the ergostane triterpenoid compound to obtain the stereoisomer by using a high performance liquid chromatography column and under a condition of a solvent of an acetonitrile and an acid-containing water in a mobile phase.

8. A method for detecting a first amount of a stereoisomer of at least one ergostane triterpenoid compound in a fruiting body of an Antrodia cinnamomea, comprising steps of:
extracting from the fruiting body an ethyl acetate extract;
detecting the ethyl acetate extract by using a ¹H nuclear magnetic resonance (¹H NMR) to identify whether the at least one ergostane triterpenoid compound is present in the ethyl acetate extract; and
detecting the first amount of the stereoisomer of the at least one ergostane triterpenoid compound in the ethyl acetate extract by using a high performance liquid chromatography (HPLC) when the at least one ergostane triterpenoid compound is present in the ethyl acetate extract.

9. The method according to claim 8, wherein the ethyl acetate extract is obtained by sequentially extracting the fruiting body of A. cinnamomea with an ethanol solution, an n-hexane solution and an ethyl acetate solution.

10. The method according to claim 8, wherein the at least one ergostane triterpenoid compound has a methylene signal at a C-28 position, and the method further comprises a step of detecting the methylene signal by using the ¹H NMR.

11. The method according to claim 8 further used to detect at least one lanostane triterpenoid compound having a second amount in the fruiting body and comprising steps of:
detecting the ethyl acetate extract by using the ¹H NMR to identify whether the at least one lanostane triterpenoid compound is present in the ethyl acetate extract; and
detecting the second amount by using the HPLC when the at least one lanostane triterpenoid compound is present in the ethyl acetate extract.

12. The method according to claim 11, wherein the at least one lanostane triterpenoid compound has a methylene signal at a C-28 position, and the method further comprises a step of detecting the methylene signal by using the ¹H NMR.

13. The method according to claim 12, wherein the HPLC comprises a detector, and the detector is one selected from a group consisting of a full wavelength detector, a single wavelength detector, a tandem mass spectrometer and a combination thereof.

14. A method for isolating a stereoisomer of a compound having a pKa value and an asymmetrical center at an α-position of a carboxylic group, comprising steps of:
calculating the pKa value being represented by a symbol A;
adjusting a pH value of a separating solvent to have a value B ranged at A−1.5≦B≦A+1.5 and 1.0≦B≦7; and
chromatographing the compound by using the separating solvent to isolate the stereoisomer.

15. A method for detecting an amount of an ergostane triterpenoid compound having a methylene signal at a C-28 position in an extract, comprising steps of:
preparing a nuclear magnetic resonance spectrum and a calibration curve based on zhankuic acid A samples with a plurality of concentrations;
detecting the methylene signal at the C-28 position by using a ¹H nuclear magnetic resonance; and
comparing the calibration curve with the methylene signal at the C-28 position to calculate the amount by an integral area ratio of the methylene signal at the C-28 position.

16. A method for detecting an amount of a lanostane triterpenoid compound having a methylene signal at a C-28 position in an extract, comprising steps of:
preparing a nuclear magnetic resonance spectrum and a calibration curve based on dehydroeburicoic acid samples with a plurality of concentrations;
detecting the methylene signal at the C-28 position by using a ¹H nuclear magnetic resonance; and
comparing the calibration curve with the methylene signal at the C-28 position to calculate the amount by an integral area ratio of the methylene signal at the C-28 position.

17. A method for detecting a stereoisomer of an ergostane triterpenoid compound in an ethyl acetate extract of a fruiting body of Antrodia cinnamomea, comprising steps of:
chromatographing the ethyl acetate extract by using a high performance liquid chromatography (HPLC) column to isolate the stereoisomer; and
determining one of an R-form and an S-form at a C-25 position of the stereoisomer according to a ¹H nuclear magnetic resonance (¹H NMR) spectrum of the stereoisomer, a retention time of the HPLC column and an optical rotation.
